SEQUENCE LISTING (60 sequences)

SEQ ID NO: 1 - protein, 6 aa, synthetic construct
  REGION 1..6: note = Synthetic Construct
SGYYWN

SEQ ID NO: 2 - protein, 16 aa, synthetic construct
  REGION 1..16: note = Synthetic Construct
YISYSGSNNYNPSLKN

SEQ ID NO: 3 - protein, 7 aa, synthetic construct
  REGION 1..7: note = Synthetic Construct
GGYYFDY

SEQ ID NO: 4 - protein, 17 aa, synthetic construct
  REGION 1..17: note = Synthetic Construct
KSSQSLLNTNNQKNYLA

SEQ ID NO: 5 - protein, 7 aa, synthetic construct
  REGION 1..7: note = Synthetic Construct
FASTRES

SEQ ID NO: 6 - protein, 9 aa, synthetic construct
  REGION 1..9: note = Synthetic Construct
QQHYNAPRT

SEQ ID NO: 7 - protein, 116 aa, synthetic construct
  REGION 1..116: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSS

SEQ ID NO: 8 - protein, 113 aa, synthetic construct
  REGION 1..113: note = Synthetic Construct
DIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIK

SEQ ID NO: 9 - protein, 446 aa, synthetic construct
  REGION 1..446: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 446 aa, synthetic construct
  REGION 1..446: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 220 aa, synthetic construct
  REGION 1..220: note = Synthetic Construct
DIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 5 aa, synthetic construct
  REGION 1..5: note = Synthetic Construct
IYGMS

SEQ ID NO: 13 - protein, 17 aa, synthetic construct
  REGION 1..17: note = Synthetic Construct
TISRDASYTYFPDSVKG

SEQ ID NO: 14 - protein, 12 aa, synthetic construct
  REGION 1..12: note = Synthetic Construct
LGDNDRGYAMDY

SEQ ID NO: 15 - protein, 16 aa, synthetic construct
  REGION 1..16: note = Synthetic Construct
RSSQSVVHVNANTYLE

SEQ ID NO: 16 - protein, 7 aa, synthetic construct
  REGION 1..7: note = Synthetic Construct
KVSNRFP

SEQ ID NO: 17 - protein, 9 aa, synthetic construct
  REGION 1..9: note = Synthetic Construct
FQGSHVPRT

SEQ ID NO: 18 - protein, 121 aa, synthetic construct
  REGION 1..121: note = Synthetic Construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSS

SEQ ID NO: 19 - protein, 112 aa, synthetic construct
  REGION 1..112: note = Synthetic Construct
DVVMTQSPLSLPVTLGQPASISCRSSQSVVHVNANTYLEWYQQRPGQSPRLLIYKVSNRFPGVPDRFSGSGSGTDFTLKISRVEAEDVGVYYCFQGSHVPRTFGGGTKVEIK

SEQ ID NO: 20 - protein, 451 aa, synthetic construct
  REGION 1..451: note = Synthetic Construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 451 aa, synthetic construct
  REGION 1..451: note = Synthetic Construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 219 aa, synthetic construct
  REGION 1..219: note = Synthetic Construct
DVVMTQSPLSLPVTLGQPASISCRSSQSVVHVNANTYLEWYQQRPGQSPRLLIYKVSNRFPGVPDRFSGSGSGTDFTLKISRVEAEDVGVYYCFQGSHVPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 15 aa, synthetic construct
  REGION 1..15: note = Synthetic Construct
GGGGSGGGGSGGGGS

SEQ ID NO: 24 - protein, 5 aa, synthetic construct
  REGION 1..5: note = Synthetic Construct
SNCMG

SEQ ID NO: 25 - protein, 17 aa, synthetic construct
  REGION 1..17: note = Synthetic Construct
VICTGGGSPSYADSVKG

SEQ ID NO: 26 - protein, 16 aa, synthetic construct
  REGION 1..16: note = Synthetic Construct
DLLRAGTPLSSYEFNY

SEQ ID NO: 27 - protein, 125 aa, synthetic construct
  REGION 1..125: note = Synthetic Construct
QVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 28 - protein, 586 aa, synthetic construct
  REGION 1..586: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 29 - protein, 220 aa, synthetic construct; identical to SEQ ID NO: 11
  REGION 1..220: note = Synthetic Construct

SEQ ID NO: 30 - protein, 586 aa, synthetic construct
  REGION 1..586: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 31 - protein, 220 aa, synthetic construct; identical to SEQ ID NO: 11
  REGION 1..220: note = Synthetic Construct

SEQ ID NO: 32 - protein, 586 aa, synthetic construct
  REGION 1..586: note = Synthetic Construct
QVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSSGGGGSGGGGSGGGGSQVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 220 aa, synthetic construct; identical to SEQ ID NO: 11
  REGION 1..220: note = Synthetic Construct

SEQ ID NO: 34 - protein, 446 aa, synthetic construct; identical to SEQ ID NO: 9
  REGION 1..446: note = Synthetic Construct

SEQ ID NO: 35 - protein, 360 aa, synthetic construct
  REGION 1..360: note = Synthetic Construct
QVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSSGGGGSGGGGSGGGGSDIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 591 aa, synthetic construct
  REGION 1..591: note = Synthetic Construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 37 - protein, 219 aa, synthetic construct; identical to SEQ ID NO: 22
  REGION 1..219: note = Synthetic Construct

SEQ ID NO: 38 - protein, 591 aa, synthetic construct
  REGION 1..591: note = Synthetic Construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 39 - protein, 219 aa, synthetic construct; identical to SEQ ID NO: 22
  REGION 1..219: note = Synthetic Construct

SEQ ID NO: 40 - protein, 220 aa, synthetic construct
  REGION 1..220: note = Synthetic Construct
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 41 - protein, 232 aa, synthetic construct
  REGION 1..232: note = Synthetic Construct
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 42 - protein, 444 aa, synthetic construct
  REGION 1..444: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSSLSPG

SEQ ID NO: 43 - protein, 445 aa, synthetic construct
  REGION 1..445: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 585 aa, synthetic construct
  REGION 1..585: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 45 - protein, 585 aa, synthetic construct
  REGION 1..585: note = Synthetic Construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 46 - protein, 220 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 47 - protein, 220 aa, synthetic construct
CPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 48 - protein, 220 aa, synthetic construct
CPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 49 - protein, 220 aa, synthetic construct
CPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 50 - protein, 220 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 51 - protein, 220 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 52 - protein, 220 aa, synthetic construct
CPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 53 - protein, 220 aa, synthetic construct
CPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 54 - protein, 220 aa, synthetic construct
CPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 55 - protein, 220 aa, synthetic construct
CPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 56 - protein, 220 aa, synthetic construct
CPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 57 - protein, 4 aa, synthetic construct
  REGION 1..4: note = Synthetic Construct
  VARIANT 3..4: note = Can be absent or present in repeats of up to 7
GSGS

SEQ ID NO: 58 - protein, 4 aa, synthetic construct
  REGION 1..4: note = Synthetic Construct
  VARIANT 1..4: note = Can be present in repeats of 0-8
GGGS

SEQ ID NO: 59 - protein, 5 aa, synthetic construct
  REGION 1..5: note = Synthetic Construct
  VARIANT 1..5: note = Can be present in repeats of 0-8
GGGGS

SEQ ID NO: 60 - protein, 5 aa, synthetic construct
  REGION 1..5: note = Synthetic Construct
  VARIANT 1..5: note = Can be present in repeats of 0-8
GSGGS

(19) United States
(12) Patent Application Publication
(10) Pub. No.: US 2025/0074995 A1
(43) Pub. Date: Mar. 6, 2025

(54) MULTISPECIFIC CONSTRUCTS AND USES THEREOF

(71) Applicant: I-Mab Biopharma Co., Ltd., Shanghai (CN)

(72) Inventors: Wenqing JIANG, Shanghai (CN); Jian LI, Shanghai (CN); Xi CHEN, Shanghai (CN); Yan LIU, Shanghai (CN); Bingshi GUO, Shanghai (CN)

(73) Assignee: I-Mab Biopharma Co., Ltd., Shanghai (CN)

(21) Appl. No.: 18/727,561

(22) PCT Filed: Jan. 9, 2023
(86) PCT No.: PCT/CN2023/071334

§ 371 Date: Jul. 9, 2024

(30) Foreign Application Priority Data
Jan. 9, 2022 (WO) PCT/CN2022/070870
Sep. 6, 2022 (WO) PCT/CN2022/117334

(51) Int. Cl. C07K 16/28; A61K 39/00; A61P 35/00

(52) U.S. Cl. CPC C07K 16/2878; A61P 35/00; A61K 2039/505; C07K 2317/31; C07K 2317/565; C07K 2317/569

(57) ABSTRACT

Provided are bispecific and multi-specific antibodies that target claudin 6 (CLDN6) and 4-1BB. These antibodies, in the absence of CLDN6-expressing cells, can bind to 4-1BB but are unable to activate 4-1BB signaling. In the presence of CLDN6-expressing cells, however, these antibodies can trigger CLDN6-dependent 4-1BB signaling, leading to potent immune response to the CLDN6-expressing tumor cells.

### CROSS REFERENCE TO RELATED APPLICATIONS

This application claims priority to International Application No. PCT/CN2022/070870, filed Jan. 9, 2022, the contents content of which are incorporated herein by reference in their entirety.

### FIELD OF THE APPLICATION

The present application relates to multispecific molecules, e.g., anti-CLDN6/anti-4-1BB bispecific antibodies, and uses thereof including treating diseases or conditions.

### BACKGROUND OF THE APPLICATION

Claudins are a family of proteins that form the important components of the tight cell junctions. Claudin-6 (CLDN6) is a tetraspan membrane protein involved in the formation of tight junctions. In the adult human normal tissues, CLDN6 mRNA and protein is absent. On the other hand, high CLDN6 transcript levels are frequently detected in various human solid cancers such as testicular, ovarian, uterine and lung adenocarcinoma. In line with mRNA level, CLDN6 proteins are reported to be high and homogenous in these human cancers.

4-1BB (CD137, tumor necrosis factor receptor superfamily 9) is a member of TNF-receptor superfamily (TNFRSF) and is a costimulatory molecule which is expressed following the activation of immune cells, both innate and adaptive immune cells. 4-1BB plays an important role in modulating the activity of various immune cells. 4-1BB agonists enhance immune cell proliferation, survival, secretion of cytokines and cytolytic activity CD8 T cells. Many other studies showed that activation of 4-1BB enhances immune response to eliminate tumors in mice. Therefore, it was suggested that 4-1BB is a promising target molecule in cancer immunology.

The disclosures of all publications, patents, patent applications and published patent applications referred to herein are hereby incorporated herein by reference in their entirety.

### BRIEF SUMMARY OF THE APPLICATION

In one aspect, provided herein is a multispecific construct comprising: a first antibody moiety that specifically binds to a tumor antigen; and a second antibody moiety that specifically binds to 4-1BB, wherein binding of the first antibody moiety with the tumor antigen triggers the second antibody moiety to activate 4-1BB. In some embodiments, the second antibody moiety specifically binds to CRD 3/4 region of 4-1BB.

In some embodiments, provided herein is a multispecific construct comprising a first antibody moiety that specifically binds to a tumor antigen; and a second antibody moiety that specifically binds to 4-1BB, wherein binding of the first antibody moiety with the tumor antigen triggers the second antibody moiety to activate 4-1BB. In some embodiments, the activation of 4-1BB by the second antibody moiety is enhanced by at least about any one of 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety with the tumor antigen, including any range in between these values. In some embodiments, without binding to the tumor antigen, the multispecific construct does activate 4-1BB signaling. In some embodiments, without binding to the tumor antigen, the second moiety does not activate 4-1BB signaling.

In some embodiments, provided are multispecific constructs comprising a) a first antibody moiety that specifically binds to claudin-6 (“CLDN6”); and b) a second antibody moiety that specifically binds to 4-1BB. In some embodiments, the first antibody moiety comprises a full-length antibody comprising two heavy chains and two light chains.

Provided herein is a multispecific construct comprising: (1) a first antibody moiety that specifically binds to claudin-6 (“CLDN6”); and (2) a second antibody moiety that specifically binds to 4-1BB.

In some embodiments, the first antibody moiety is selected from the group consisting of a full-length antibody, Fab, Fab′, F(ab′)₂, scFv, and sdAb. In some embodiments, wherein the first antibody moiety comprises: (1) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 7; and (2) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID No: 8. In some embodiments, the first antibody moiety comprises a heavy variable region (VH) and a light chain variable region (VL), wherein: (a) the VH comprises: (i) a HC-CDR1 comprising an amino acid sequence of SEQ ID NO: 1, (ii) a HC-CDR2 comprising an amino acid sequence of SEQ ID NO: 2, and (iii) a HC-CDR3 comprising an amino acid sequence of SEQ ID NO: 3, and (b) the VL comprises: (i) a LC-CDR1 comprising an amino acid sequence of SEQ ID NO: 4, (ii) a LC-CDR2 comprising an amino acid sequence of SEQ ID NO: 5, and (iii) a LC-CDR3 comprising an amino acid sequence of SEQ ID NO: 6. In some embodiments, the first antibody moiety comprises a heavy variable region (VH) and a light chain variable region (VL), and wherein: (1) the VH comprises the amino acid sequence of SEQ ID NO: 7, or a variant thereof comprising at least about 80% sequence identity to SEQ ID NO: 7; and/or (2) the VL comprises the amino acid sequence of SEQ ID NO: 8, or a variant thereof comprising at least about 80% sequence identity to SEQ ID NO: 8. In some embodiments, the first antibody moiety comprises: (1) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 18; and (2) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID N): 19. In some embodiments, the first antibody moiety comprises a heavy variable region (VH) and a light chain variable region (VL), wherein: (1) the VH comprises: (i) a HC-CDR1 comprising an amino acid sequence of SEQ ID NO: 12, (ii) a HC-CDR2 comprising an amino acid sequence of SEQ ID NO: 13, and (iii) a HC-CDR3 comprising an amino acid sequence of SEQ ID NO: 14, and; (2) the VL comprises: (i) a LC-CDR1 comprising an amino acid sequence of SEQ ID NO: 15, (ii) a LC-CDR2 comprising an amino acid sequence of SEQ ID NO: 16, and (iii) a LC-CDR3 comprising an amino acid sequence of SEQ ID NO: 17. In some embodiments, the first antibody moiety comprises a heavy variable region (VH) and a light chain variable region (VL), and wherein: (1) the VH comprises the amino acid sequence of SEQ ID NO: 18, or a variant thereof having at least about 80% sequence identity to SEQ ID NO: 18; and/or (2) the VL comprises the amino acid sequence of SEQ ID NO: 19, or a variant thereof having at least about 80% sequence identity to SEQ ID NO: 19. In some embodiments, binding of the first antibody moiety with CLDN6 triggers the second antibody moiety to activate 4-1BB.

In some embodiments, the second antibody moiety specifically binds to CRD 3/4 region of 4-1BB. In some embodiments, the second antibody moiety is selected from the group consisting of a full-length antibody, Fab, Fab′, F(ab′)₂, scFv, and sdAb. In some embodiments, the second antibody moiety is a sdAb. In some embodiments, the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27. In some embodiments, the sdAb comprises: (1) a sdAb-CDR1 comprising an amino acid sequence of SEQ ID NO: 24; (2) a sdAb-CDR2 comprising an amino acid sequence of SEQ ID NO: 25; and (3) a sdAb-CDR3 comprising an amino acid sequence of SEQ ID NO: 26. In some embodiments, the second antibody moiety comprises the amino acid sequence of SEQ ID NO:27, or a variant thereof having at least about 80% sequence identity to SEQ ID NOs: 27.

In some embodiments, the multispecific construct is a bispecific antibody or a bispecific binding fragment. In some embodiments, (1) the first antibody moiety comprises: (a) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 7; (b) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID No: 8, and (2) the second antibody moiety comprises a sdAb-CDR1, a sdAb-CDR2, and a sdAb CDR3, respectively comprising the amino acid sequence of a CDR1, a CDR2, and a CDR3 within a single monomeric variable antibody domain comprising the amino acid sequence set forth in SEQ ID NOs: 27.

In some embodiments, provided is a pharmaceutical composition comprising a multispecific construct described herein and a pharmaceutical acceptable carrier.

In some embodiments, provided is a nucleic acid encoding a multispecific construct described herein. In some embodiments, provided is a vector comprising such nucleic acid. In some embodiments, provided us a host cell comprising such nucleic acid or such vector.

Also provided herein is a method of treating a disease or condition in a subject in need thereof, comprising administering to the subject an effective amount of comprising a multispecific construct described herein or a pharmaceutical composition described herein. Also provided herein is a use of a multispecific construct described herein in the preparation of a medicament for treating a disease or a condition in a subject in need thereof. In some embodiments, the disease or condition is cancer.

In some embodiments, provided is a multispecific construct comprising: (1) a first antibody moiety that specifically binds to a tumor antigen; and (2) a second antibody moiety that specifically binds to 4-1BB, wherein binding of the first antibody moiety with the tumor antigen triggers the second antibody moiety to activate 4-1BB. In some embodiments, the activation of 4-1BB by the second antibody moiety is enhanced by at least 10-fold after binding of the first antibody moiety with the tumor antigen. In some embodiments, the second antibody moiety specifically binds to CRD 3/4 region of 4-1BB. In some embodiments, the second antibody moiety is a sdAb. In some embodiments, the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27. In some embodiments, the sdAb comprises: (1) a sdAb-CDR1 comprising an amino acid sequence of SEQ ID NO: 24; (2) a sdAb-CDR2 comprising an amino acid sequence of SEQ ID NO: 25; and (3) a sdAb-CDR3 comprising an amino acid sequence of SEQ ID NO: 26. In some embodiments, the tumor antigen is CLDN6.

Also provided herein is a multispecific construct, comprising two heavy chain components and two light chain components, wherein: (a) each heavy chain component comprises a sequence set forth in SEQ ID NO: 28 and/or SEQ ID NO: 44 and each light chain component comprises a sequence set forth in SEQ ID NO: 29; (b) each heavy chain component comprises a sequence set forth in SEQ ID NO: 30 and each light chain component comprises a sequence set forth in SEQ ID NO: 31; (c) each heavy chain component comprises a sequence set forth in SEQ ID NO: 32 and each light chain component comprises a sequence set forth in SEQ ID NO: 33; (d) each heavy chain component comprises a sequence set forth in SEQ ID NO: 34 and each light chain component comprises a sequence set forth in SEQ ID NO: 35; (e) each heavy chain component comprises a sequence set forth in SEQ ID NO: 36 and each light chain component comprises a sequence set forth in SEQ ID NO: 37; or (f) each heavy chain component comprises a sequence set forth in SEQ ID NO: 38 and each light chain component comprises a sequence set forth in SEQ ID NO: 39.

The present application provides kits comprising a multispecific construct described herein and/or a pharmaceutical described herein and a package insert or instructions for using the multispecific construct (or pharmaceutical composition) for treating a disease or condition (e.g., cancer).

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1A-1B illustrate binding affinity of CLDN6×4-1BB bispecific antibodies (BsAbs) disclosed herein with CLDN6.

FIG. 2A-2C illustrate binding affinity of CLDN6×4-1BB BsAbs disclosed herein with CLDN6 expressing cells.

FIG. 3A-3D illustrate binding affinity of CLDN6×4-1BB BsAbs disclosed herein with 4-1BB.

FIG. 4A-4B illustrate binding of CLDN6×4-1BB BsAbs disclosed herein with soluble 4-1BB and cells expressing 4-1BB.

FIG. 5A-5D illustrate CLDN6-dependent 4-1BB activation of CLDN6×4-1BB BsAbs disclosed herein.

FIG. 6A-6H illustrate activation of PBMC by CLDN6×4-1BB BsAbs disclosed herein.

FIG. 7A-7B illustrate tumor inhibition in hu4-1BB mice bearing CT26 murine colorectal carcinoma tumors after treatment with CLDN6×4-1BB BsAbs disclosed herein.

FIG. 8 illustrates liver function after treatment of the CLDN6×4-1BB BsAbs of the present application.

FIG. 9A-9B illustrate tumor inhibition in hu4-1BB mice bearing MC38 murine colon adenocarcinoma tumors after treatment with CLDN6×4-1BB BsAbs disclosed herein.

### DETAILED DESCRIPTION OF THE APPLICATION

### Definitions

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (al heavy chain), or IgA2 (α2 heavy chain).

The term “antigen-binding fragment” as used herein refers to an antibody fragment including, for example, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a single domain antibody (e.g., a camelized single domain antibody), a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Single-chain Fv,” also abbreviated as “sFv” or “scFv,” are antibody fragments that comprise the VH and VL antibody domains connected into a single polypeptide chain. In some embodiments, the scFv polypeptide further comprises a polypeptide linker between the VH and VL domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR Definitions

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra with minor modification. Briefly, we added 5 more residues in super variable loop before the heavy chain CDR1. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C3” domain) comprises the stretch of residues C-terminal to a CH2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fe receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731.

As use herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BIACORE™-tests and peptide scans.

An “isolated” antibody (or construct) is one that has been identified, separated and/or recovered from a component of its production environment (e.g., natural or recombinant). Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

The terms “agonizing” or “agonize” refer to an increase or enhancement of any phenotypic characteristic, or the incidence, degree, or likelihood of that characteristic. To “increase” or “enhance” is to decrease, reduce or arrest an activity, function, and/or amount as compared to that of a reference. In certain embodiments, by “increase” or “enhance” is meant the ability to cause an overall increase in, e.g., activity, function, and/or amount, of at least about 1-fold or greater. In another embodiment, by “increase” or “enhance” is meant the ability to cause an overall increase in, e.g., activity, function, and/or amount, of at least about 5-fold or greater as compared to a reference. In yet another embodiment, by “increase” or “enhance” is meant the ability to cause an overall increase in, e.g., activity, function, and/or amount, of at least about any one of 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold, as compared to a reference, including any range in between these values, or greater than about 100-fold, as compared to a reference.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing” as used herein, includes providing prophylaxis with respect to the occurrence or recurrence of a disease in an individual that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

As used herein, “based upon” includes assessing, determining, or measuring the individual's characteristics as described herein (and preferably selecting an individual suitable for receiving treatment). When the status of a claudin-18 aberration is “used as a basis” for selection, assessing, measuring, or determining method of treatment as described herein, the CLDN6 aberration determined before and/or during treatment, and the status (including presence, absence, expression level, activity level and/or phosphorylation level of CLDN6) obtained is used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

The terms “subject,” “individual,” and “patient” are used interchangeably herein to refer to a mammal, including, but not limited to, human, bovine, horse, feline, canine, rodent, or primate. In some embodiments, the individual is a human.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### Antibody Binding Affinity

Binding specificity of the antibody moieties of the multispecific constructs described herein can be determined experimentally by methods known in the art. Such methods comprise, but are not limited to Western blots, ELISA-, RIA-, ECL-, IRMA-, EIA-, BIACORE™-tests and peptide scans.

In some embodiments, the binding affinity is measured by the dissociation constant, K_{D}. Dissociation constants may be determined through any analytical technique known in the art, including biochemical or biophysical techniques such as fluorescent activated cell sorting (FACS), flow cytometry, enzyme-linked immunosorbent assay (ELISA), surface plasmon resonance (SPR), BioLayer interferometry (see, e.g., Octet System by ForteBio), meso scale discover assays (see, e.g., MSD-SET), isothermal titration calorimetry (ITC), differential scanning calorimetry (DSC), circular dichroism (CD), stopped-flow analysis, and colorimetric or fluorescent protein melting analyses; or a cell binding assay.

In some embodiments, the K_{D} of the binding between the antibody moiety and the target antigen (e.g., CLDN6 or 4-1BB) is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and the target antigen (e.g., CLDN6 or 4-1BB) is stronger than about any one of 10⁻⁷ M, 10⁻⁸ M, 10⁻⁹ M, 10⁻¹⁰ M, 10⁻¹¹ M, or 10⁻¹² M. In some embodiments, the target antigen (e.g., CLDN6 or 4-1BB) is a human antigen.

In some embodiments, the Kₒₙ of the binding between the antibody moiety and the target antigen (e.g., CLDN6 or 4-1BB) is about 10³ M⁻¹ s⁻¹ to about 10⁸ M⁻¹ s⁻¹, about 10³ M⁻¹ s⁻¹ to about 10⁴ M⁻¹ s⁻¹, about 10⁴ M⁻¹ s⁻ to about 10⁵ M⁻¹ s⁻¹, about 10⁵ M⁻¹ s⁻¹, to about 10⁶ M⁻¹ s⁻¹, about 10⁶ M⁻¹ s⁻¹ to about 10⁷ M⁻¹ s⁻¹, or about 10⁷ M⁻¹ s⁻¹ to about 10⁸ M⁻¹ s⁻¹. In some embodiments, the Kₒₙ of the binding between the antibody moiety and the target antigen (e.g., CLDN6 or 4-1BB) is about 10³ M⁻¹ s⁻¹ to about 10⁵ M⁻¹ s⁻¹, about 10⁴ M⁻¹ s⁻¹ to about 10⁶ M⁻¹ s⁻¹, about 10⁵ M⁻¹ s⁻¹ to about 10⁷ M⁻¹ s⁻¹, about 10⁶ M⁻¹ s⁻¹ to about 10⁸ M⁻¹ s⁻¹, about 10⁴ M⁻¹ s⁻ to about 10⁷ M⁻¹ s⁻¹, or about 10⁵ M⁻¹ s to about 10⁸ M⁻¹ s⁻¹. In some embodiments, the Kₒₙ of the binding between the antibody moiety and the target antigen (e.g., CLDN6 or 4-1BB) is no more than about any one of 10³ M⁻¹ s⁻¹, 10⁴ M⁻¹ s⁻¹, 10⁵ M⁻¹ s⁻¹, 10⁶ M⁻¹ s⁻¹, 10⁷ M⁻¹ s⁻¹ or 10⁸ M⁻¹ s⁻¹. In some embodiments, the target antigen (e.g., CLDN6 or 4-1BB) is human antigen.

In some embodiments, the K_{off} of the binding between the antibody moiety and the target antigen (e.g., CLDN6 or 4-1BB) is about 1 s to about 10⁻⁶ s⁻¹, about 1 s to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻ to about 10⁻⁶ s⁻¹, about 1 s to about 10⁻⁵ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s⁻¹. In some embodiments, the K_{off} of the binding between the antibody moiety and the target antigen (e.g., CLDN6 or 4-1BB) is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹. In some embodiments, the target antigen (e.g., CLDN6 or 4-1BB) is human antigen.

### Chimeric or Humanized Antibodies

In some embodiments, one or more of the antibody moieties of the multispecific constructs of the present application is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In some embodiments, one or more of the antibody moieties of the multispecific constructs of the present application is a human antibody (known as human domain antibody, or human DAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

### Substitution, Insertion, and Deletion Variants

In some embodiments, antibody variants comprising one or more amino acid substitutions are included in the multispecific constructs described herein. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding decreased immunogenicity or improved ADCC or CDC.

TABLE 2
Amino acid substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant VHH sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Glycosylation Variants

In some embodiments, one or more antibody moieties of the multispecific construct of the present application is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody moiety comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the antibody moiety has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the antibody moiety has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### Fc Region Variants

In some embodiments, one or more amino acid modifications may be introduced into the Fc region of the antibody moiety, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the Fc fragment possesses some but not all effector functions, which make it a desirable candidate for applications in which the half-life of the antibody moiety in vivo is important yet certain effector functions (such as complement dependent cytotoxicity (CDC) and antibody-dependent cellular cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056), wherein amino acid numbering is according to the EU Index. See, e.g., Kabat et al. (1991) Sequences of Proteins of Immunological Interest, Fifth Edition, U.S. Department of Health and Human Services, NIH Publication No. 91-3242). Such Fc mutants include Fc mutants with substitutions at two or more of amino acid positions 265, 269, 270, 297 and 327 (wherein amino acid numbering is according to the EU index), including the so-called “DANA” Fc mutant with substitution of residues 265 and 297 to alanine (U.S. Pat. No. 7,332,581) wherein amino acid numbering is according to the EU Index.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the Fc fragment is an IgG1 Fc fragment. In some embodiments, the IgG1 Fc fragment comprises a L234A mutation and/or a L235A mutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the antibody moiety variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### Cysteine Engineered Antibody Variants

In some embodiments, it may be desirable to create cysteine engineered antibody moieties, e.g., “thioMAbs,” in which one or more residues of one or more of the antibody moieties in a multispecific construct herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

### Anti-CLDN6 Antibody Moieties

The anti-CLDN6 antibody moieties of the multispecific constructs described in the present application include any antibody moieties that specifically bind to claudin-6 (“CLDN6”).

### Claudin 6 (CLDN6)

Claudins are a family of tight junction membrane proteins that are expressed in epithelia and endothelia and form paracellular barriers and pores that determine tight junction permeability. Claudins have been recognized as crucial regulators in the initiation, progression, and metastasis of cancers, playing distinct roles in a variety of cancers according to their different patterns of tissue-dependent expression (Tabaries et al. (2017). “The role of claudins in cancer metastasis.” Oncogene 36, 1176-1190). Claudin-6 (CLDN6) is a member of the claudin family and serves as a tight junction molecule, which plays a vital role in cell-to-cell adhesion in epithelial or endothelial cell sheets. It encodes the tetraspan membrane protein, with the size of 220 amino acids and molecular mass of 23,292 Da. CLDN6 has been identified to be the origination of cell adhesion signaling taking part in the regulation of nuclear receptor activity through targeting molecules of the nuclear receptor superfamily and managing their gene expression (Sugimoto et al. (2019). “Cell adhesion signals regulate the nuclear receptor activity.” Proc. Natl. Acad. Sci. U.S.A. 116, 24600-24609). CLDN6 appears to be significantly upregulated in 20 types of human cancers (Zhang et al. (2021) Front. Cell. Dev. Biol. 9: 726656). In some embodiments, the CLDN6 is a human CLDN6 (“hCLDN6”). In some embodiments, the hCLDN6 comprises an amino acid sequence set forth in SEQ ID NO: 40 or a variant thereof (e.g., a post translationally modified variant and/or conformation variant).

### Exemplary Anti-CLDN6 Antibody Moieties

In some embodiments, the anti-CLDN6 antibody moiety competes for binding to CLDN6 with an antibody moiety comprising a) a heavy variable region (V_{H}) and a light chain variable region (V_{L}), wherein: a) the V_{H} comprises: i) a HC-CDR1 comprising an amino acid sequence of SEQ ID NO: 1, ii) a HC-CDR2 comprising an amino acid sequence of SEQ ID NO: 2, and iii) a HC-CDR3 comprising an amino acid sequence of SEQ ID NO: 3, and; b) the V_{L} comprises: i) a LC-CDR1 comprising an amino acid sequence of SEQ ID NO: 4, ii) a LC-CDR2 comprising an amino acid sequence of SEQ ID NO: 5, and iii) a LC-CDR3 comprising an amino acid sequence of SEQ ID NO: 6. In some embodiments, the anti-CLDN6 antibody moiety competes for binding to CLDN6 with an antibody moiety comprising a) a heavy variable region (V_{H}) and a light chain variable region (V_{L}), wherein: a) the V_{H} comprises: i) a HC-CDR1 comprising an amino acid sequence of SEQ ID NO: 12, ii) a HC-CDR2 comprising an amino acid sequence of SEQ ID NO: 13, and iii) a HC-CDR3 comprising an amino acid sequence of SEQ ID NO: 14, and; b) the V_{L} comprises: i) a LC-CDR1 comprising an amino acid sequence of SEQ ID NO: 15, ii) a LC-CDR2 comprising an amino acid sequence of SEQ ID NO: 16, and iii) a LC-CDR3 comprising an amino acid sequence of SEQ ID NO: 17.

The anti-CLDN6 antibody moieties described in the present application include any antibody moieties that specifically bind to CLDN6. In some embodiments, the anti-CLDN6 antibody moiety of the present application comprises: a) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 7, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to the sequence set forth in SEQ ID NO: 7; and b) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID NO: 8, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to the sequence set forth in SEQ ID NO: 8. In some embodiments, the affinity of such anti-CLDN6 antibody moiety for CLDN6 (e.g., human CLDN6) is comparable (e.g., the same as) to that that of an anti-CLDN6 antibody moiety comprising a CDR1, a CDR2, and a CDR3 within a heavy variable region (VH) comprising the sequence set forth in SEQ ID No: 7, and a CDR1, a CDR2, and a CDR3 within a light chain variable region (VL) comprising the sequence set forth in SEQ ID NO: 8.

In some embodiments, the anti-CLDN6 antibody moiety comprises a) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 18, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to the sequence set forth in SEQ ID NO: 18; and b) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID NO: 19, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to the sequence set forth in SEQ ID NO: 19. In some embodiments, the affinity of such anti-CLDN6 antibody moiety for CLDN6 (e.g., human CLDN6) is comparable (e.g., the same as) to that that of an anti-CLDN6 antibody moiety comprising a CDR1, a CDR2, and a CDR3 within a heavy variable region (VH) comprising the sequence set forth in SEQ ID No: 18, and a CDR1, a CDR2, and a CDR3 within a light chain variable region (VL) comprising the sequence set forth in SEQ ID NO: 19.

In some embodiments, the anti-CLDN6 antibody moiety comprises a heavy variable region (VH) and a light chain variable region (VL), wherein: a) the VH comprises: i) a HC-CDR1 comprising an amino acid sequence of SEQ ID NO: 1, or a variant thereof comprising up to about 3 (such as any of about 1, 2, 3) amino acid substitutions in the HC-CDR1, ii) a HC-CDR2 comprising an amino acid sequence of SEQ ID NO: 2, or a variant thereof comprising up to about 3 (such as any of about 1, 2, 3) amino acid substitutions in the HC-CDR2, and iii) a HC-CDR3 comprising an amino acid sequence of SEQ ID NO: 3, or a variant thereof comprising up to about 3 (such as any of about 1, 2, 3) amino acid substitutions in the HC-CDR3, and; b) the VL comprises: i) a LC-CDR1 comprising an amino acid sequence of SEQ ID NO: 4, or a variant thereof comprising up to about 3 (such as any of about 1, 2, 3) amino acid substitutions in the LC-CDR1, ii) a LC-CDR2 comprising an amino acid sequence of SEQ ID NO: 5, or a variant thereof comprising up to about 3 (such as any of about 1, 2, 3) amino acid substitutions in the LC-CDR2, and iii) a LC-CDR3 comprising an amino acid sequence of SEQ ID NO: 6, or a variant thereof comprising up to about 3 (such as any of about 1, 2, 3) amino acid substitutions in the LC-CDR3.

In some embodiments, the anti-CLDN6 antibody moiety comprises a heavy variable region (VH) and a light chain variable region (VL), wherein: a) the VH comprises: i) a HC-CDR1 comprising an amino acid sequence of SEQ ID NO: 12, or a variant thereof comprising up to about 3 (such as any of about 1, 2, 3) amino acid substitutions in the HC-CDR1, ii) a HC-CDR2 comprising an amino acid sequence of SEQ ID NO: 13, or a variant thereof comprising up to about 3 (such as any of about 1, 2, 3) amino acid substitutions in the HC-CDR2, and iii) a HC-CDR3 comprising an amino acid sequence of SEQ ID NO: 14, or a variant thereof comprising up to about 3 (such as any of about 1, 2, 3) amino acid substitutions in the HC-CDR3, and; b) the VL comprises: i) a LC-CDR1 comprising an amino acid sequence of SEQ ID NO: 15, or a variant thereof comprising up to about 3 (such as any of about 1, 2, 3) amino acid substitutions in the LC-CDR1, ii) a LC-CDR2 comprising an amino acid sequence of SEQ ID NO: 16, or a variant thereof comprising up to about 3 (such as any of about 1, 2, 3) amino acid substitutions in the LC-CDR2, and iii) a LC-CDR3 comprising an amino acid sequence of SEQ ID NO: 17, or a variant thereof comprising up to about 3 (such as any of about 1, 2, 3) amino acid substitutions in the LC-CDR3.

In some embodiments, the anti-CLDN6 antibody moiety comprises a heavy variable region (VH) and a light chain variable region (VL), wherein: the VH comprises the amino acid sequence of SEQ ID NO: 7, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to SEQ ID NO: 7; and/or the VL comprises the amino acid sequence of SEQ ID NO: 8, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to SEQ ID NO: 8. In some embodiments, the affinity of such anti-CLDN6 antibody moiety for CLDN6 (e.g., human CLDN6) is comparable (e.g., the same as) to that that of an anti-CLDN6 antibody moiety comprising a VH comprising the sequence set forth in SEQ ID No: 7, and a VL comprising the sequence set forth in SEQ ID NO: 8.

In some embodiments, the anti-CLDN6 antibody moiety comprises a heavy variable region (VH) and a light chain variable region (VL), wherein: the VH comprises the amino acid sequence of SEQ ID NO: 18, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to SEQ ID NO: 18; and/or the VL comprises the amino acid sequence of SEQ ID NO: 19, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to SEQ ID NO: 19. In some embodiments, the affinity of such anti-CLDN6 antibody moiety for CLDN6 (e.g., human CLDN6) is comparable (e.g., the same as) to that that of an anti-CLDN6 antibody moiety comprising a VH comprising the sequence set forth in SEQ ID No: 18, and a VL comprising the sequence set forth in SEQ ID NO: 19.

The anti-CLDN6 antibody moiety of the present application can be any suitable format known in the art. In some embodiments, the anti-CLDN6 antibody moiety can be selected from the group consisting of full-length antibody, Fab, Fab′, F(ab′)2, scFv, and sdAb.

In some embodiments, the anti-CLDN6 antibody moiety comprises a full-length antibody comprising two heavy chains and two light chains. In some embodiments, the full-length antibody has an Fc fragment selected from the group consisting of Fc fragments from IgG, IgA, IgD, IgE, IgM, and combinations and hybrids thereof. In some embodiments, the Fc fragment is selected from the group consisting of Fc fragments from IgG1, IgG2, IgG3, IgG4, and combinations and hybrids thereof. In some embodiments, the Fc fragment is an IgG1 or IgG4 Fc fragment.

In some embodiments, the Fc fragment has a reduced FcγR binding affinity as compared to a wild type Fc. In some embodiments, the Fc fragment comprises one or more substitutions selected from the group consisting of N297A, N297Q, N297G, or L235E. In some embodiments, the Fc fragment is an IgG1 fragment comprising one or more substitutions selected from the group consisting of N297A, N297Q, N297G, L235E, and/or L234A/L235A. In some embodiments, the Fc fragment is an IgG1 fragment comprising N297A. In some embodiments, the Fc fragment is an IgG4 fragment comprising N297A, N297Q, N297G, L235E, and/or F234A/L235A. In some embodiments, the Fc fragment is an IgG4 fragment comprising N297A. In some embodiment, the Fc fragment comprises an amino sequence as set forth in SEQ ID NO: 42.

In some embodiments, the anti-CLDN6 antibody moiety comprises a full-length antibody comprising two heavy chains and two light chains, wherein the heavy chain comprises the amino acid sequence of SEQ ID NO: 9 or SEQ ID NO: 42, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to SEQ ID NO: 9 or SEQ ID NO: 42; and/or the light chain comprises the amino acid sequence of SEQ ID NO: 11 or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to SEQ ID NO: 11. In some embodiments, the affinity of such anti-CLDN6 antibody moiety for CLDN6 (e.g., human CLDN6) is comparable (e.g., the same as) to that that of an anti-CLDN6 antibody moiety comprising two heavy chains, each comprising SEQ ID NO: 9 and/or SEQ ID NO: 42, and two light chains, each comprising SEQ ID NO: 11.

In some embodiments, the anti-CLDN6 antibody moiety comprises a full-length antibody comprising two heavy chains and two light chains, wherein the heavy chain comprises the amino acid sequence of SEQ ID NO: 10 or SEQ ID NO: 43, or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to SEQ ID NO: 10 or SEQ ID NO: 43; and/or the light chain comprises the amino acid sequence of SEQ ID NO: 11 or a variant thereof having at least about 80% (including, for example, at least about any one of 80%, 85%, 87%, 89%, 90% 91%, 92%, 93%, 94%, 95%, 96%, 97%, 98%, or 99%, or more than 99%) sequence identity to SEQ ID NO: 11. In some embodiments, the affinity of such anti-CLDN6 antibody moiety for CLDN6 (e.g., human CLDN6) is comparable (e.g., the same as) to that that of an anti-CLDN6 antibody moiety comprising two heavy chains, each comprising SEQ ID NO: 10 and/or SEQ ID NO: 43, and two light chains, each comprising SEQ ID NO: 11.

In some embodiments, the anti-CLDN6 antibody moiety comprises a full-length antibody comprising two heavy chains and two light chains, wherein the heavy chain comprises the amino acid sequence of SEQ ID NO: 20, or a variant thereof comprising at least about 80% (including for example at least about any of 80%, 85%, 90%, 95%, 96%, 97%, 98%, or 99%) sequence identity to SEQ ID NO: 20; and/or the light chain comprises the amino acid sequence of SEQ ID NO: 22 or a variant thereof comprising at least about 80% (including for example at least about any of 80%, 85%, 90%, 95%, 96%, 97%, 98%, or 99%) sequence identity to SEQ ID NO: 22. In some embodiments, the affinity of such anti-CLDN6 antibody moiety for CLDN6 (e.g., human CLDN6) is comparable (e.g., the same as) to that that of an anti-CLDN6 antibody moiety comprising two heavy chains, each comprising SEQ ID NO: 20, and two light chains, each comprising SEQ ID NO: 22.

In some embodiments, the anti-CLDN6 antibody moiety comprises a full-length antibody comprising two heavy chains and two light chains, wherein the heavy chain comprises the amino acid sequence of SEQ ID NO: 21, or a variant thereof having at least about 80% (including for example at least about any of 80%, 85%, 90%, 95%, 96%, 97%, 98%, or 99%) sequence identity to SEQ ID NO: 21; and/or the light chain comprises the amino acid sequence of SEQ ID NO: 22 or a variant thereof having at least about 80% (including for example at least about any of 80%, 85%, 90%, 95%, 96%, 97%, 98%, or 99%) sequence identity to SEQ ID NO: 22. In some embodiments, the affinity of such anti-CLDN6 antibody moiety for CLDN6 (e.g., human CLDN6) is comparable (e.g., the same as) to that that of an anti-CLDN6 antibody moiety comprising two heavy chains, each comprising SEQ ID NO: 21, and two light chains, each comprising SEQ ID NO: 22.

### Anti-4-1BB Antibody Moiety

The anti-4-1BB antibody moieties of the multispecific constructs described in the present application include any antibody moieties that specifically bind to 4-1BB. In some embodiments, the 4-1BB is a human 4-1BB (“h4-1BB”). h4-1BB is a type I transmembrane receptor with four extracellular cysteine-rich domains (“CRDs”, i.e., CRD1, CDR2, CRD3, and CRD4) followed by a short transmembrane domain and a C-terminal cytoplasmic region. CRD2 and CRD3 of h4-1BB interact with the ligand 4-1BBL (Bitra et al. (2018) J Biol Chem. 293(26): 9958-9969. In contrast to other TNFRs, h4-1BB exists as a disulfide-linked dimer, and Dimerization likely occurs through an unpaired cysteine (Cys¹²¹) found within CRD4 of h4-1BB. In some embodiments, the h4-1BB comprises the sequence set forth in SEQ ID NO: 41 or a variant thereof (e.g., a post translationally modified variant and/or conformation variant). In some embodiments, the anti-4-1BB antibody moiety binds to the CRD3/CRD4 region of 4-1BB.

The anti-4-1BB antibody moiety can be any suitable format known in the art. In some embodiments, the anti-4-1BB antibody moiety is selected from the group consisting of full-length antibody, Fab, Fab′, F(ab′)₂, scFv, and sdAb. In some embodiments, the anti-4-1BB antibody moiety comprises a single-domain antibody that binds to 4-1BB.

### Exemplary Anti-4-1BB Antibody Moieties

In some embodiments, the anti-4-1BB antibody moiety comprises a single domain antibody (sdAb) comprising a sdAb-CDR1, a sdAb-CDR2, and a sdAb-CDR3, respectively comprising the amino acid sequence of a CDR1, a CDR2, and a CDR3 within a single monomeric variable antibody domain having the amino acid sequence set forth in SEQ ID NO: 27, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27.

In some embodiments, the anti-4-1BB antibody moiety comprises a single domain antibody (sdAb) comprising: a) a sdAb-CDR1 comprising an amino acid sequence of SEQ ID NO: 24, or a variant thereof comprising up to about 3 (such as any of about 1, 2, 3) amino acid substitutions in the sdAb-CDR1; b) a sdAb-CDR2 comprising an amino acid sequence of SEQ ID NO: 25, or a variant thereof comprising up to about 3 (such as any of about 1, 2, 3) amino acid substitutions in the sdAb-CDR2; and c) a sdAb-CDR3 comprising an amino acid sequence of SEQ ID NO: 26, or a variant thereof comprising up to about 3 (such as any of about 1, 2, 3) amino acid substitutions in the sdAb-CDR3.

In some embodiments, the anti-4-1BB antibody moiety comprises a single domain antibody (sdAb) comprising a) a sdAb-CDR1 comprising an amino acid sequence of SEQ ID NO: 24, a sdAb-CDR2 comprising an amino acid sequence of SEQ ID NO: 25, and a sdAb-CDR3 comprising an amino acid sequence of SEQ ID NO: 26.

In some embodiments, the anti-4-1BB antibody moiety comprises a single domain antibody (sdAb) comprising the amino acid sequence of SEQ ID NOs: 27, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27.

### Multispecific Constructs

In one aspect, provided herein is a multispecific construct comprising: a first antibody moiety that specifically binds to a tumor antigen; and a second antibody moiety that specifically binds to 4-1BB, wherein binding of the first antibody moiety with the tumor antigen triggers the second antibody moiety to activate 4-1BB. In some embodiments, the second antibody moiety specifically binds to CRD 3/4 region of 4-1BB.

In some embodiments, provided herein is a multispecific construct comprising a first antibody moiety that specifically binds to a tumor antigen; and a second antibody moiety that specifically binds to 4-1BB, wherein binding of the first antibody moiety with the tumor antigen triggers the second antibody moiety to activate 4-1BB. In some embodiments, the activation of 4-1BB by the second antibody moiety is enhanced by at least about any one of 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety with the tumor antigen, including any range in between these values. In some embodiments, without binding to the tumor antigen, the multispecific construct does activate 4-1BB signaling. In some embodiments, without binding to the tumor antigen, the second moiety does not activate 4-1BB signaling.

In some embodiments of the multispecific construct of the present application, the second antibody moiety is a sdAb. In some embodiments, the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27.

In some embodiments, the sdAb comprises a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3.

In some embodiments, the sdAb comprises the amino acid sequence of SEQ ID NO:27, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 27. In some embodiments, the affinity of such sdAb for 4-1BB (e.g., human 4-1BB) is comparable (e.g., the same as) to that that of an sdAb comprising SEQ ID NO: 27.

The tumor antigen that the first antibody moiety specifically binds to can be any suitable tumor antigen known in the art. In some embodiments, the tumor antigen is CLDN6.

In some embodiments, the present application provides multispecific constructs that bind to both CLDN6 and 4-1BB. In some embodiments, the multispecific construct described herein is a bispecific antibody comprising an anti-CLDN6 antibody moiety and an anti-4-1BB antibody moiety. Anti-CLDN6 antibody moieties and anti-4-1BB antibody moieties can be any of those described herein.

In some embodiments of the multispecific construct of the present application, the multispecific construct described herein is a multispecific construct (e.g., a bispecific antibody) comprising an anti-CLDN6 antibody moiety and an anti-4-1BB antibody moiety, wherein the anti-CLDN6 antibody moiety comprises: (1) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 7, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to SEQ ID NO: 7; and (2) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID No: 8, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to SEQ ID NO: 8. In some embodiments, the affinity of such anti-CLDN6 antibody moiety for CLDN6 (e.g., human CLDN6) is comparable (e.g., the same as) to that that of an anti-CLDN6 antibody moiety comprising a CDR-1, a CDR-2 and a CDR-3 of the VH set forth in SEQ ID NO: 7 and a CDR-1, a CDR-2 and a CDR-3 of the VL set forth in SEQ ID NO: 8. In some embodiments, the anti-4-1BB antibody moiety comprises a sdAb comprising: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the anti-4-1BB antibody moiety comprises an sdAb comprising the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27.

In some embodiments, the multispecific construct described herein is a multispecific construct (e.g., a bispecific antibody) comprising an anti-CLDN6 antibody moiety and an anti-4-1BB antibody moiety, wherein the anti-CLDN6 antibody moiety comprises a heavy variable region (VH) and a light chain variable region (VL), wherein the VH comprises: (i) a HC-CDR1 comprising an amino acid sequence of SEQ ID NO: 1, or a variant thereof comprising up to about 3 (such as any of about 1, 2, 3) amino acid substitutions in the HC-CDR1, (ii) a HC-CDR2 comprising an amino acid sequence of SEQ ID NO: 2, or a variant thereof comprising up to about 3 (such as any of about 1, 2, 3) amino acid substitutions in the HC-CDR2; and (iii) a HC-CDR3 comprising an amino acid sequence of SEQ ID NO: 3, or a variant thereof comprising up to about 3 (such as any of about 1, 2, 3) amino acid substitutions in the HC-CDR3; and the VL comprises: (i) a LC-CDR1 comprising an amino acid sequence of SEQ ID NO: 4, or a variant thereof comprising up to about 3 (such as any of about 1, 2, 3) amino acid substitutions in the LC-CDR1; (ii) a LC-CDR2 comprising an amino acid sequence of SEQ ID NO: 5, or a variant thereof comprising up to about 3 (such as any of about 1, 2, 3) amino acid substitutions in the LC-CDR2; and (iii) a LC-CDR3 comprising an amino acid sequence of SEQ ID NO: 6, or a variant thereof comprising up to about 3 (such as any of about 1, 2, 3) amino acid substitutions in the LC-CDR3. In some embodiments, the anti-4-1BB antibody moiety comprises a sdAb comprising: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the anti-4-1BB antibody moiety comprises an sdAb comprising the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27.

In some embodiments, the multispecific construct described herein is a multispecific construct (e.g., a bispecific antibody) comprising an anti-CLDN6 antibody moiety and an anti-4-1BB antibody moiety, wherein the anti-CLDN6 antibody moiety comprises the VH comprises the amino acid sequence of SEQ ID NO: 7, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to SEQ ID NO: 7; and/or the VL comprises the amino acid sequence of SEQ ID NO: 8, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to SEQ ID NO: 8. In some embodiments, the affinity of such anti-CLDN6 antibody moiety for CLDN6 (e.g., human CLDN6) is comparable (e.g., the same as) to that that of an anti-CLDN6 antibody moiety comprising a VH set forth in SEQ ID NO: 7 and a VL set forth in SEQ ID NO: 8. In some embodiments, the anti-4-1BB antibody moiety comprises a sdAb comprising: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the anti-4-1BB antibody moiety comprises an sdAb comprising the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27.

In some embodiments, the multispecific construct described herein is a multispecific construct (e.g., a bispecific antibody) comprising an anti-CLDN6 antibody moiety and an anti-4-1BB antibody moiety, wherein the anti-CLDN6 antibody moiety comprises: (1) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 18, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to SEQ ID NO: 18; and (2) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID No: 19, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to SEQ ID NO: 19. In some embodiments, the affinity of such anti-CLDN6 antibody moiety for CLDN6 (e.g., human CLDN6) is comparable (e.g., the same as) to that that of an anti-CLDN6 antibody moiety comprising a CDR-1, a CDR-2 and a CDR-3 of the VH set forth in SEQ ID NO: 18 and a CDR-1, a CDR-2 and a CDR-3 of the VL set forth in SEQ ID NO: 19. In some embodiments, the anti-4-1BB antibody moiety comprises a sdAb comprising: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the anti-4-1BB antibody moiety comprises an sdAb comprising the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27.

In some embodiments, the multispecific construct described herein is a multispecific construct (e.g., a bispecific antibody) comprising an anti-CLDN6 antibody moiety and an anti-4-1BB antibody moiety, wherein the anti-CLDN6 antibody moiety comprises a heavy variable region (VH) and a light chain variable region (VL), wherein the VH comprises: (i) a HC-CDR1 comprising an amino acid sequence of SEQ ID NO: 12, or a variant thereof comprising up to about 3 (such as any of about 1, 2, 3) amino acid substitutions in the HC-CDR1, (ii) a HC-CDR2 comprising an amino acid sequence of SEQ ID NO: 13, or a variant thereof comprising up to about 3 (such as any of about 1, 2, 3) amino acid substitutions in the HC-CDR2, and (iii) a HC-CDR3 comprising an amino acid sequence of SEQ ID NO: 14, or a variant thereof comprising up to about 3 (such as any of about 1, 2, 3) amino acid substitutions in the HC-CDR3; and the VL comprises: (i) a LC-CDR1 comprising an amino acid sequence of SEQ ID NO: 15, or a variant thereof comprising up to about 3 (such as any of about 1, 2, 3) amino acid substitutions in the LC-CDR1; (ii) a LC-CDR2 comprising an amino acid sequence of SEQ ID NO: 16, or a variant thereof comprising up to about 3 (such as any of about 1, 2, 3) amino acid substitutions in the LC-CDR2; and (iii) a LC-CDR3 comprising an amino acid sequence of SEQ ID NO: 17, or a variant thereof comprising up to about 3 (such as any of about 1, 2, 3) amino acid substitutions in the LC-CDR3. In some embodiments, the anti-4-1BB antibody moiety comprises a sdAb comprising: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the anti-4-1BB antibody moiety comprises an sdAb comprising the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27.

In some embodiments of the multispecific construct of the present application, the multispecific construct described herein is a multispecific construct (e.g., a bispecific antibody) comprising an anti-CLDN6 antibody moiety and an anti-4-1BB antibody moiety, wherein the anti-CLDN6 antibody moiety comprises the VH comprises the amino acid sequence of SEQ ID NO: 18, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to SEQ ID NO: 18; and/or the VL comprises the amino acid sequence of SEQ ID NO: 19, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to SEQ ID NO: 19. In some embodiments, the affinity of such anti-CLDN6 antibody moiety for CLDN6 (e.g., human CLDN6) is comparable (e.g., the same as) to that that of an anti-CLDN6 antibody moiety comprising a VH set forth in SEQ ID NO: 18 and a VL set forth in SEQ ID NO: 19. In some embodiments, the anti-4-1BB antibody moiety comprises a sdAb comprising: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the anti-4-1BB antibody moiety comprises an sdAb comprising the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27.

In some embodiments of the multispecific construct of the present application, the multispecific construct described herein is a multispecific construct (e.g., a bispecific antibody) comprising an anti-CLDN6 antibody moiety and an anti-4-1BB antibody moiety, wherein the anti-CLDN6 antibody moiety comprises the VH comprises the amino acid sequence of SEQ ID NO: 18; and/or the VL comprises the amino acid sequence of SEQ ID NO: 19. In some embodiments, the anti-4-1BB antibody moiety comprises a sdAb comprising: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the anti-4-1BB antibody moiety comprises an sdAb comprising the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27.

In some embodiments, the multispecific construct described herein is a multispecific construct (e.g., a bispecific antibody) comprising an anti-CLDN6 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-CLDN6 antibody moiety and an anti-4-1BB antibody moiety, and the anti-4-1BB antibody moiety comprises a sdAb.

In some embodiments, the multispecific construct described herein is a multispecific construct (e.g., a bispecific antibody) comprising an anti-CLDN6 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27.

In some embodiments, the multispecific construct described herein is a multispecific construct (e.g., a bispecific antibody) comprising an anti-CLDN6 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3.

In some embodiments, the multispecific construct described herein is a multispecific construct (e.g., a bispecific antibody) comprising an anti-CLDN6 antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27.

In some embodiments, there is provided a multispecific construct (e.g., bispecific antibody) that comprises an anti-CLDN6 antibody moiety comprising an antibody (e.g., a full-length antibody) specifically binds to CLDN6 (e.g., human CLDN6) and an anti-4-1BB antibody moiety comprising an antibody (e.g., a single domain antibody) that binds to 4-1BB (e.g., human 4-1BB), wherein the anti-CLDN6 antibody moiety comprises: (1) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 7; and (2) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID No: 8; and the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27.

In some embodiments, there is provided a multispecific construct (e.g., bispecific antibody) that comprises an anti-CLDN6 antibody moiety comprising an antibody (e.g., a full-length antibody) specifically binds to CLDN6 (e.g., human CLDN6) and an anti-4-1BB antibody moiety comprising an antibody (e.g., a single domain antibody) that binds to 4-1BB (e.g., human 4-1BB), wherein the anti-CLDN6 antibody moiety comprises: (1) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 18; and (2) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID NO: 19; and the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27.

In some embodiments, there is provided a multispecific construct (e.g., bispecific antibody) that comprises an anti-CLDN6 antibody moiety comprising an antibody (e.g., a full-length antibody) specifically binds to CLDN6 (e.g., human CLDN6) and an anti-4-1BB antibody moiety comprising an antibody (e.g., a single domain antibody) that binds to 4-1BB (e.g., human 4-1BB), wherein the anti-CLDN6 antibody moiety comprises the VH comprises the amino acid sequence of SEQ ID NO: 7, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 7; and/or the VL comprises the amino acid sequence of SEQ ID NO: 8, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 8; and the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such multispecific construct (e.g., bispecific antibody) for CLDN6 and 4-1BB is comparable (e.g., the same as) to that that of a multispecific construct comprising an anti-CLDN6 antibody moiety comprising a full-length antibody that comprises a VH set forth in SEQ ID NO: 7 and a VL set forth in SEQ ID NO:8 and an anti-4-1BB antibody moiety comprising a single domain antibody comprising the sequence set forth in SEQ ID NO: 27.

In some embodiments, there is provided a multispecific construct (e.g., bispecific antibody) that comprises an anti-CLDN6 antibody moiety comprising an antibody (e.g., a full-length antibody) specifically binds to CLDN6 (e.g., human CLDN6) and an anti-4-1BB antibody moiety comprising an antibody (e.g., a single domain antibody) that binds to 4-1BB (e.g., human 4-1BB), wherein the anti-CLDN6 antibody moiety comprises the VH comprises the amino acid sequence of SEQ ID NO: 18, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 18; and/or the VL comprises the amino acid sequence of SEQ ID NO: 19, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 19; and the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such multispecific construct (e.g., bispecific antibody) for CLDN6 and 4-1BB is comparable (e.g., the same as) to that that of a multispecific construct comprising an anti-CLDN6 antibody moiety comprising a full-length antibody that comprises a VH set forth in SEQ ID NO: 18 and a VL set forth in SEQ ID NO: 19 and an anti-4-1BB antibody moiety comprising a single domain antibody comprising the sequence set forth in SEQ ID NO: 27.

In some embodiments, there is provided a multispecific construct (e.g., bispecific antibody) that comprises an anti-CLDN6 antibody moiety comprising an antibody (e.g., a full-length antibody) that specifically binds to CLDN6 (e.g., human CLDN6) and an anti-4-1BB antibody moiety that specifically binds 4-1BB, wherein the anti-4-1BB antibody moiety is fused to the N-terminus of the one or both heavy chains of the anti-CLDN6 antibody (e.g., full-length anti-CLDN6 antibody). In some embodiments, there is provided a multispecific construct (e.g., bispecific antibody) that comprises an anti-CLDN6 antibody moiety comprising an antibody (e.g., a full-length antibody) that specifically binds to CLDN6 and an anti-4-1BB antibody moiety that specifically binds 4-1BB, wherein the anti-4-1BB antibody moiety is fused to the C-terminus of the one or both heavy chains of the anti-CLDN antibody (e.g., the full-length anti-CLDN6 antibody). In some embodiments, there is provided a multispecific construct (e.g., bispecific antibody) that comprises an anti-CLDN6 antibody moiety comprising an antibody (e.g., a full-length antibody) that specifically binds to CLDN6 and an anti-4-1BB antibody moiety that specifically binds 4-1BB, wherein the anti-4-1BB antibody moiety is fused to the N-terminus of the one or both light chains of the anti-CLDN6 antibody (e.g., the full-length anti-CLDN6 antibody). In some embodiments, there is provided a multispecific construct (e.g., bispecific antibody) that comprises an anti-CLDN6 antibody moiety comprising an antibody (e.g., a full-length antibody) that specifically binds to CLDN6 and an anti-4-1BB antibody moiety, wherein the anti-4-1BB antibody moiety is fused to the C-terminus of the one or both light chains of the anti-CLDN6 antibody (e.g., the full-length anti-CLDN6 antibody).

In some embodiments, there is provided a multispecific construct (e.g., bispecific antibody) that comprises an anti-CLDN6 antibody moiety comprising an antibody (e.g., a full-length antibody) that specifically binds to CLDN6 (e.g., human CLDN6) and an anti-4-1BB antibody moiety comprising a single domain antibody that binds to 4-1BB (e.g., human 4-1BB), wherein the single domain antibody is fused to the N-terminus of the one or both heavy chains of the anti-CLDN antibody (e.g., full-length anti-CLDN antibody). In some embodiments, there is provided a multispecific construct (e.g., bispecific antibody) that comprises an anti-CLDN6 antibody moiety comprising an antibody (e.g., a full-length antibody) that specifically binds to CLDN6 and an anti-4-1BB antibody moiety comprising a single domain antibody that binds to 4-1BB, wherein the single domain antibody is fused to the C-terminus of the one or both heavy chains of the anti-CLDN antibody (e.g., the full-length anti-CLDN antibody). In some embodiments, there is provided a multispecific construct (e.g., bispecific antibody) that comprises an anti-CLDN6 antibody moiety comprising an antibody (e.g., a full-length antibody) that specifically binds to CLDN6 and an anti-4-1BB antibody moiety comprising a single domain antibody that binds to 4-1BB, wherein the single domain antibody is fused to the N-terminus of the one or both light chains of the anti-CLDN6 antibody (e.g., the full-length anti-CLDN6 antibody). In some embodiments, there is provided a multispecific construct (e.g., bispecific antibody) that comprises an anti-CLDN6 antibody moiety comprising an antibody (e.g., a full-length antibody) that specifically binds to CLDN6 and an anti-4-1BB antibody moiety comprising a single domain antibody that binds to 4-1BB, wherein the single domain antibody is fused to the C-terminus of the one or both light chains of the anti-CLDN6 antibody (e.g., the full-length anti-CLDN6 antibody).

In some embodiments, the anti-4-1BB antibody moiety is fused to the anti-CLDN6 antibody moiety via a linker. In some embodiments, the linker is a peptide linker. In some embodiments, the linker has a length of about four to about fifty amino acids. In some embodiments, the linker is selected from the group consisting of (GS)n, (GGGS)n, (GGGGS)n, and (GSGGS)n. In some embodiments, the n is 0-8. In some embodiments, the linker comprises an amino acid sequence of GGGGSGGGGSGGGGS.

In some embodiments, provided herein is a multispecific construct comprising a heavy chain component and a light chain component, wherein the heavy chain component comprises the amino acid sequence of SEQ ID NO: 28, 30, 32, 34, 36, 38, 44, or 45, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 28, 30, 32, 34, 36, 38, 44 or 45; and/or the light chain comprises the amino acid sequence of SEQ ID NO: 29, 31, 33, 35, 37, 39, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 29, 31, 33, 35, 37, 39.

In some embodiments, provided herein is a multispecific construct comprising a heavy chain component and a light chain component, wherein the heavy chain component comprises the amino acid sequence of SEQ ID NO: 28, 30, 32, 34, 36, 38, 44, or 45; and/or the light chain comprises the amino acid sequence of SEQ ID NO: SEQ ID NO: 29, 31, 33, 35, 37, 39.

In some embodiments, provided is a multispecific construct comprising two heavy chain components and two light chain components, wherein: (a) each heavy chain component comprises a sequence set forth in SEQ ID NO: 28 and/or 44 and each light chain component comprises a sequence set forth in SEQ ID NO: 29; (b) each heavy chain component comprises a sequence set forth in SEQ ID NO: 30 and/or SEQ ID NO: 45 and each light chain component comprises a sequence set forth in SEQ ID NO: 31; (c) each heavy chain component comprises a sequence set forth in SEQ ID NO: 32 and each light chain component comprises a sequence set forth in SEQ ID NO: 33; (d) each heavy chain component comprises a sequence set forth in SEQ ID NO: 34 and each light chain component comprises a sequence set forth in SEQ ID NO: 35; (e) each heavy chain component comprises a sequence set forth in SEQ ID NO: 36 and each light chain component comprises a sequence set forth in SEQ ID NO: 37; or (f) each heavy chain component comprises a sequence set forth in SEQ ID NO: 38 and each light chain component comprises a sequence set forth in SEQ ID NO: 39.

In some embodiments, provided herein is a multispecific construct comprising a first antibody moiety that specifically binds to CLDN6; and a second antibody moiety that specifically binds to 4-1BB, wherein binding of the first antibody moiety with CLDN6 triggers the second antibody moiety to activate 4-1BB. In some embodiments, the activation of 4-1BB by the second antibody moiety is enhanced by at least 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety with CLDN6. In some embodiments, without binding to CLDN6, the multispecific construct does not activate 4-1BB signaling. In some embodiments, without binding to CLDN6, the second moiety does activate 4-1BB signaling.

4-1BB signaling activation is the expected mechanism for agonist antibodies, such as utomilumab (PF-05082566) and urelumab (BMS-663513). The anti-4-1BB portions of some of the presently disclosed antibodies, however, do not require such an activity. Actually, in some embodiments, it is preferred that the anti-4-1BB portions of the present antibodies are not capable of independently activating 4-1BB in the absence of CLDN6 binding. As the experimental examples demonstrated, interestingly, when the anti-CLDN6 portion binds to CLDN6 proteins on a cell, such CLDN6 binding can trigger 4-1BB signaling activation.

Compared to the known anti-4-1BB agonist antibodies which are commonly associated with dose-limiting on-target hepatotoxicities, the antibodies of the present disclosure are contemplated to be much safer. In a tissue, such as liver, wherein CLDN6 is not expressed in the healthy condition, the antibodies of the present disclosure are not expected to trigger cytotoxic immune response as they cannot activate 4-1BB signaling. In a tumor tissue wherein CLDN6 is expressed and/or accessible, by contrast, the present antibodies can initiate potent immune response to the tumor cells. Accordingly, unlike those anti-4-1BB antibodies currently being developed clinically which suffer on-target/inherent toxicities, the presently disclosed antibodies can be potent and safe at the same time in treating cancer.

### Nucleic Acids

Nucleic acid molecules encoding the multispecific constructs or various antibody moieties described herein are also contemplated. In some embodiments, there is provided a nucleic acid (or a set of nucleic acids) encoding one or more polypeptides of the multispecific constructs or various antibody moieties. In some embodiments, there is provided a nucleic acid (or a set of nucleic acids) encoding a multispecific construct (e.g., an anti-CLDN6/anti-4-1BB bispecific antibody), or polypeptide portion thereof.

Also contemplated here are isolated host cell comprising a multispecific construct (e.g., an anti-CLDN6/anti-4-1BB bispecific antibody), nucleic acid(s) encoding the polypeptide components of the multispecific construct, or a vector comprising a nucleic acid encoding the polypeptide components of the multispecific construct (e.g., an anti-CLDN6/anti-4-1BB bispecific antibody) described herein.

The present application also includes variants to these nucleic acid sequences. For example, the variants include nucleotide sequences that hybridize to the nucleic acid sequences encoding the multispecific construct (e.g., an anti-CLDN6/anti-4-1BB bispecific antibody) or various antibody moieties described herein under at least moderately stringent hybridization conditions.

The present application also provides vectors in which a nucleic acid of the present application is inserted.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

### Methods of Treatment

Also provided here are methods of treating a disease or condition in an individual. The methods comprise administering a multispecific construct (e.g., an anti-CLDN6/anti-4-1BB bispecific antibody) described herein into individuals (e.g., mammals such as humans). In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc.

In some embodiments of the method, the disease or condition is a proliferative disorder. In some embodiments the cell proliferative disorder is cancer. In some embodiments, the cancer is solid tumor, melanoma, renal cancer, ovarian cancer, colorectal cancer, Squamous cell carcinoma of head and neck (SCCHN), non-small cell lung cancer, or non-Hodgkin lymphoma (NHL).

### Compositions, Kits and Articles of Manufacture

Also provided herein are compositions (such as formulations) comprising any one of the multispecific constructs (e.g., an anti-CLDN6/anti-4-1BB bispecific antibody) described herein, a nucleic acid encoding any of the multispecific constructs or a portion thereof, a vector comprising the nucleic acid encoding one of the multispecific constructs, or a host cell comprising the nucleic acid or vector.

Suitable formulations of the multispecific construct (e.g., an anti-CLDN6/anti-4-1BB bispecific antibody) described herein can be obtained by mixing the multispecific construct having the desired degree of purity with optional pharmaceutically acceptable carriers, excipients or stabilizers (Remington's Pharmaceutical Sciences 16th edition, Osol, A. Ed. (1980)).

Also provided are kits comprising any one of the multispecific constructs (e.g., an anti-CLDN6/anti-4-1BB bispecific antibody) described herein. The kits may be useful for any of the methods of treatment described herein.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle).

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. The examples below are intended to be purely exemplary of the application and should therefore not be considered to limit the application in any way. The following examples and detailed description are offered by way of illustration and not by way of limitation.

### Example 1: Generation of CLDN6×4-1BB Bispecific Antibodies

The exemplary CLDN6×4-1BB bispecific antibodies show in Table 3 below were designed and generated.

TABLE 3
Exemplary CLDN6 x 4-1BB Bispecific Antibodies
CLDN6-1 IgG1 N297A X 4-1BB (CLDN6-1 X 4-1BB NA,
4-1BB sdAb fused to C-terminus of heavy chain of CLDN-6-1
antibody moiety, with N297A in IgG1 Fc region)

| | | |
|---|---|---|
| Heavy | Heavy chain | QVQLQESGPGLVKPSETLSLTCTVSGYSITSGYYWNWIRQPPGKGLE |
| chain | of CLDN6-1 | WMGYISYSGSNNYNPSLKNRVTISRDTSKNQFSLKLSSVTAADTAVY |
| component | (SEQ ID NO: | YCARGGYYFDYWGQGTLVTVSSASTKGPSVFPLAPSSKSTSGGTAAL |
| (SEQ ID | 10) | GCLVKDYFPEPVTVSWNSGALTSGVHTFPAVLQSSGLYSLSSVVTVPS |
| NO: 28) | | SSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVH |
| | | NAKTKPREEQYASTYRVVSVLTVLHQDWLNGKEYKCKVSNKALPAP |
| | | IEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSV |
| | | MHEALHNHYTQKSLSLSPGK |
| | Linker (SEQ | GGGGSGGGGSGGGGS |
| | ID NO: 23) | |
| | 4-1BB sdAb | QVQLQESGGGLVQPGGSLRLSCAASGYTYSSNCMGWFRQAPGKGLE |
| | (SEQ ID NO: | GVAVICTGGGSPSYADSVKGRFTISRDNAKNTLYLQMNSLRAEDTAV |
| | 27) | YYCAADLLRAGTPLSSYEFNYWGQGTLVTVSS |
| Light chain | Light chain of | DIVMTQSPDSLAVSLGERATINCKSSQSLLNTNNQKNYLAWYQQKPG |
| component | CLDN6-1 | QPPKLLIYFASTRESGVPDRFSGSGSGTDFTLTISSLQAEDVAVYFCQQ |
| (SEQ ID | (SEQ ID NO: | HYNAPRTFGGGTKVEIKRTVAAPSVFIFPPSDEQLKSGTASVVCLLNN |
| NO: 29) | 11) | FYPREAKVQWKVDNALQSGNSQESVTEQDSKDSTYSLSSTLTLSKAD |
| | | YEKHKVYACEVTHQGLSSPVTKSFNRGEC |

CLDN6-1 IgG1 WT X 4-1BB (CLDN6-1 X 4-1BB WT,
4-1BB sdAb fused to C-terminus of heavy chain of CLDN-6-1
antibody moiety, with wild type IgG1)

| | | |
|---|---|---|
| Heavy | Heavy chain | QVQLQESGPGLVKPSETLSLTCTVSGYSITSGYYWNWIRQPPGKGLE |
| chain | of CLDN6-1 | WMGYISYSGSNNYNPSLKNRVTISRDTSKNQFSLKLSSVTAADTAVY |
| component | (SEQ ID NO: | YCARGGYYFDYWGQGTLVTVSSASTKGPSVFPLAPSSKSTSGGTAAL |
| (SEQ ID | 9) | GCLVKDYFPEPVTVSWNSGALTSGVHTFPAVLQSSGLYSLSSVVTVPS |
| NO: 30) | | SSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSV |
| | | MHEALHNHYTQKSLSLSPGK |
| | Linker (SEQ | GGGGSGGGGSGGGGS |
| | ID NO: 23) | |
| | 4-1BB sdAb | QVQLQESGGGLVQPGGSLRLSCAASGYTYSSNCMGWFRQAPGKGLE |
| | (SEQ ID NO: | GVAVICTGGGSPSYADSVKGRFTISRDNAKNTLYLQMNSLRAEDTAV |
| | 27) | YYCAADLLRAGTPLSSYEFNYWGQGTLVTVSS |
| Light chain | Light chain of | DIVMTQSPDSLAVSLGERATINCKSSQSLLNTNNQKNYLAWYQQKPG |
| component | CLDN6-1 | QPPKLLIYFASTRESGVPDRFSGSGSGTDFTLTISSLQAEDVAVYFCQQ |
| (SEQ ID | (SEQ ID NO: | HYNAPRTFGGGTKVEIKRTVAAPSVFIFPPSDEQLKSGTASVVCLLNN |
| NO: 31) | 11) | FYPREAKVQWKVDNALQSGNSQESVTEQDSKDSTYSLSSTLTLSKAD |
| | | YEKHKVYACEVTHQGLSSPVTKSFNRGEC |

4-1BB X CLDN6-1 IgG1 WT (4-1BB X CLDN6-1 WT,
4-1BB sdAb fused to N-terminus of heavy chain of CLDN-6-1
antibody moiety, with wild type IgG1)

| | | |
|---|---|---|
| Heavy | 4-1BB sdAb | QVQLQESGGGLVQPGGSLRLSCAASGYTYSSNCMGWFRQAPGKGLE |
| chain | (SEQ ID NO: | GVAVICTGGGSPSYADSVKGRFTISRDNAKNTLYLQMNSLRAEDTAV |
| component | 27) | YYCAADLLRAGTPLSSYEFNYWGQGTLVTVSS |
| (SEQ ID | Linker (SEQ | GGGGSGGGGSGGGGS |
| NO: 32) | ID NO: 23) | |
| | Heavy chain | QVQLQESGPGLVKPSETLSLTCTVSGYSITSGYYWNWIRQPPGKGLE |
| | of CLDN6-1 | WMGYISYSGSNNYNPSLKNRVTISRDTSKNQFSLKLSSVTAADTAVY |
| | (SEQ ID NO: | YCARGGYYFDYWGQGTLVTVSSASTKGPSVFPLAPSSKSTSGGTAAL |
| | 9) | GCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSV |
| | | MHEALHNHYTQKSLSLSPGK |
| Light chain | Light chain of | DIVMTQSPDSLAVSLGERATINCKSSQSLLNTNNQKNYLAWYQQKPG |
| component | CLDN6 (SEQ | QPPKLLIYFASTRESGVPDRFSGSGSGTDFTLTISSLQAEDVAVYFCQQ |
| (SEQ ID | ID NO: 11) | HYNAPRTFGGGTKVEIKRTVAAPSVFIFPPSDEQLKSGTASVVCLLNN |
| NO: 33) | | FYPREAKVQWKVDNALQSGNSQESVTEQDSKDSTYSLSSTLTLSKAD |
| | | YEKHKVYACEVTHQGLSSPVTKSFNRGEC |

4-1BB X CLDN6-1 IgG1 WT (4-1BB X CLDN6-1 L WT,
4-1BB sdAb fused to N-terminus of light chain of CLDN-6-1
antibody moiety, with wild type IgG1)

| | | |
|---|---|---|
| Heavy | Heavy chain | QVQLQESGPGLVKPSETLSLTCTVSGYSITSGYYWNWIRQPPGKGLE |
| chain | of CLDN6-1 | WMGYISYSGSNNYNPSLKNRVTISRDTSKNQFSLKLSSVTAADTAVY |
| component | (SEQ ID NO: | YCARGGYYFDYWGQGTLVTVSSASTKGPSVFPLAPSSKSTSGGTAAL |
| (SEQ ID | 9) | GCLVKDYFPEPVTVSWNSGALTSGVHTFPAVLQSSGLYSLSSVVTVPS |
| NO: 34) | | SSLGTQTYICNVNHKPSNTKVDKKVEPKSCDKTHTCPPCPAPELLGGP |
| | | SVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSV |
| | | MHEALHNHYTQKSLSLSPGK |
| Light chain | 4-1BB sdAb | QVQLQESGGGLVQPGGSLRLSCAASGYTYSSNCMGWFRQAPGKGLE |
| component | (SEQ ID NO: | GVAVICTGGGSPSYADSVKGRFTISRDNAKNTLYLQMNSLRAEDTAV |
| (SEQ ID | 27) | YYCAADLLRAGTPLSSYEFNYWGQGTLVTVSS |
| NO: 35) | Linker (SEQ | GGGGSGGGGSGGGGS |
| | ID NO: 23) | |
| | Light chain of | DIVMTQSPDSLAVSLGERATINCKSSQSLLNTNNQKNYLAWYQQKPG |
| | CLDN6-1 | QPPKLLIYFASTRESGVPDRFSGSGSGTDFTLTISSLQAEDVAVYFCQQ |
| | (SEQ ID NO: | HYNAPRTFGGGTKVEIKRTVAAPSVFIFPPSDEQLKSGTASVVCLLNN |
| | 11) | FYPREAKVQWKVDNALQSGNSQESVTEQDSKDSTYSLSSTLTLSKAD |
| | | YEKHKVYACEVTHQGLSSPVTKSFNRGEC |

CLDN6-3 IgG1 N297A X 4-1BB (CLDN6-3 X 4-1BB NA,
4-1BB sdAb fused to C-terminus of heavy chain of CLDN-6-3
antibody moiety, with N297A in IgG1 Fc region)

| | | |
|---|---|---|
| Heavy | Heavy chain | EVQLVESGGGLVQPGGSLRLSCAASGFTFSIYGMSWVRQAPGKGLE |
| chain | of CLDN6-3 | WVATISRDASYTYFPDSVKGRFTISRDNAKNSLYLQMNSLRAEDTAV |
| component | (SEQ ID NO: | YYCVRLGDNDRGYAMDYWGQGTLVTVSSASTKGPSVFPLAPSSKST |
| (SEQ ID | 21) | SGGTAALGCLVKDYFPEPVTVSWNSGALTSGVHTFPAVLQSSGLYSL |
| NO: 36) | | SSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWY |
| | | VDGVEVHNAKTKPREEQYASTYRVVSVLTVLHQDWLNGKEYKCKV |
| | | SNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQ |
| | | QGNVFSCSVMHEALHNHYTQKSLSLSPGK |
| | Linker (SEQ | GGGGSGGGGSGGGGS |
| | ID NO: 23) | |
| | 4-1BB sdAb | QVQLQESGGGLVQPGGSLRLSCAASGYTYSSNCMGWFRQAPGKGLE |
| | (SEQ ID NO: | GVAVICTGGGSPSYADSVKGRFTISRDNAKNTLYLQMNSLRAEDTAV |
| | 27) | YYCAADLLRAGTPLSSYEFNYWGQGTLVTVSS |
| Light chain | Light chain of | DVVMTQSPLSLPVTLGQPASISCRSSQSVVHVNANTYLEWYQQRPGQ |
| component | CLDN6-3 | SPRLLIYKVSNRFPGVPDRFSGSGSGTDFTLKISRVEAEDVGVYYCFQ |
| (SEQ ID | (SEQ ID NO: | GSHVPRTFGGGTKVEIKRTVAAPSVFIFPPSDEQLKSGTASVVCLLNNF |
| NO: 37) | 22) | YPREAKVQWKVDNALQSGNSQESVTEQDSKDSTYSLSSTLTLSKADY |
| | | EKHKVYACEVTHQGLSSPVTKSFNRGEC |

CLDN6-3 IgG1 WT X 4-1BB (CLDN6-3 X 4-1BB WT,
4-1BB sdAb fused to C-terminus of heavy chain of CLDN-6-3
antibody moiety, with wild type IgG1)

| | | |
|---|---|---|
| Heavy | Heavy chain | EVQLVESGGGLVQPGGSLRLSCAASGFTFSIYGMSWVRQAPGKGLE |
| chain | of CLDN6-3 | WVATISRDASYTYFPDSVKGRFTISRDNAKNSLYLQMNSLRAEDTAV |
| component | (SEQ ID NO: | YYCVRLGDNDRGYAMDYWGQGTLVTVSSASTKGPSVFPLAPSSKST |
| (SEQ ID | 20) | SGGTAALGCLVKDYFPEPVTVSWNSGALTSGVHTFPAVLQSSGLYSL |
| NO: 38) | | SSVVTVPSSSLGTQTYICNVNHKPSNTKVDKKVEPKSCDKTHTCPPCP |
| | | APELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKV |
| | | SNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQ |
| | | QGNVFSCSVMHEALHNHYTQKSLSLSPGK |
| | Linker (SEQ | GGGGSGGGGSGGGGS (SEQ ID NO: 23) |
| | ID NO: 23) | |
| | 4-1BB sdAb | QVQLQESGGGLVQPGGSLRLSCAASGYTYSSNCMGWFRQAPGKGLE |
| | (SEQ ID NO: | GVAVICTGGGSPSYADSVKGRFTISRDNAKNTLYLQMNSLRAEDTAV |
| | 27) | YYCAADLLRAGTPLSSYEFNYWGQGTLVTVSS |
| Light chain | Light chain of | DVVMTQSPLSLPVTLGQPASISCRSSQSVVHVNANTYLEWYQQRPGQ |
| component | CLDN6-3 | SPRLLIYKVSNRFPGVPDRFSGSGSGTDFTLKISRVEAEDVGVYYCFQ |
| (SEQ ID | (SEQ ID NO: | GSHVPRTFGGGTKVEIKRTVAAPSVFIFPPSDEQLKSGTASVVCLLNNF |
| NO: 39) | 22) | YPREAKVQWKVDNALQSGNSQESVTEQDSKDSTYSLSSTLTLSKADY |
| | | EKHKVYACEVTHQGLSSPVTKSFNRGEC |

### Example 2. Antigen Binding Activity of the CLDN6×4-1BB BsAbs

2.1 Binding Affinity of the CLDN6×4-1BB BsAbs with CLDN6

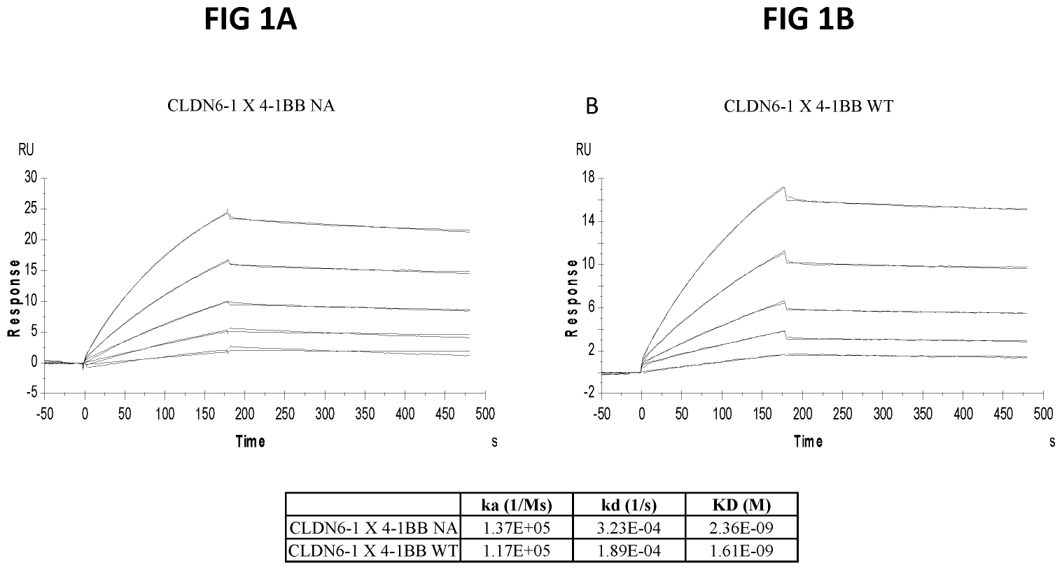

Binding affinity of CLDN6-1×4-1BB NA and CLDN6-1×4-1BB WT (as prepared in Example 1) towards human CLDN6 was measured by surface plasmon resonance (SPR). As shown in FIGS. 1A and 1B, CLDN6-1×4-1BB NA and CLDN6-1×4-1BB WT bound to CLDN6 virus-like particles (VLP) with K_{D}s of 2.36×10⁻⁹ M and 1.61×10⁻⁹ M, respectively.

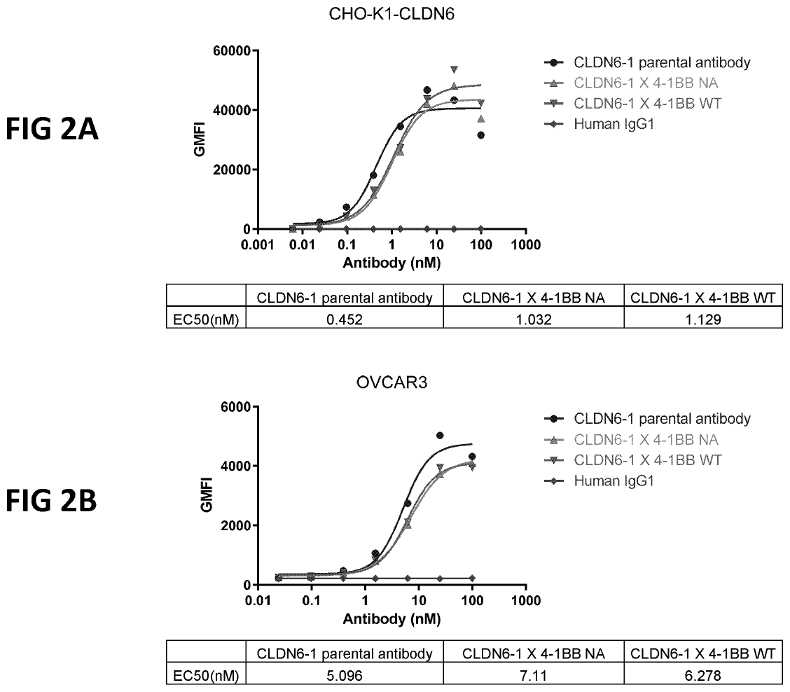

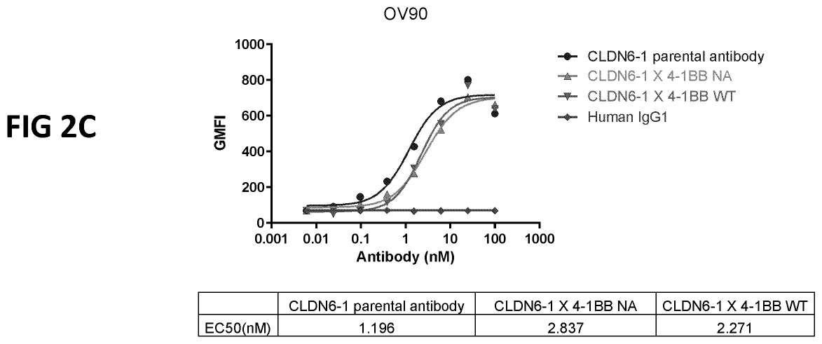

A CHO-K1 cell line stably expressing human CLDN6 (CHO-K1-CLDN6) was prepared to evaluate the binding capability of CLDN6-1×4-1BB NA and CLDN6-1×4-1BB WT towards CLDN6. The parental CLDN6-1 antibody was used as control. Briefly, CHO-K1-CLDN6 cells were incubated with the BsAbs at different concentrations for 30 minutes at 4° C. in FACS buffer. Then, phycoerythrin (PE) conjugated-anti-human IgG antibody was added after washing, and the cells were further incubated at 4° C. for 30 minutes. Mean fluorescence intensity (MFI) of PE was evaluated by FACS. As shown in FIG. 2A, CLDN6-1×4-1BB NA and CLDN6-1×4-1BB WT both bound to CLDN6-expressed cells in a concentration-dependent manner.

OVCAR3 and OV90 are human ovarian cancer cell lines with endogenous CLDN6 expression. As shown in FIGS. 2B and 2C, CLDN6-1×4-1BB NA and CLDN6-1×4-1BB WT were both capable of binding to OVCAR3 and OV90. Overall, the binding affinities of the BsAbs comprising an anti-CLDN6-1 antibody moiety for human CLDN6 are comparable to the binding affinity of the parental CLDN6-1 antibody for human CLDN6. The binding signal was well correlated with CLDN6 expression levels on the surfaces of the OVCAR3 and OV90 cells.

2.2 Binding Affinity of CLDN6×4-1BB BsAbs with 4-1BB

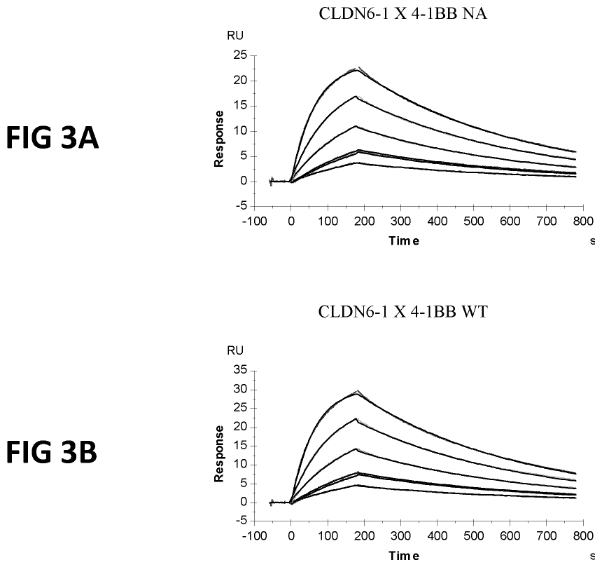

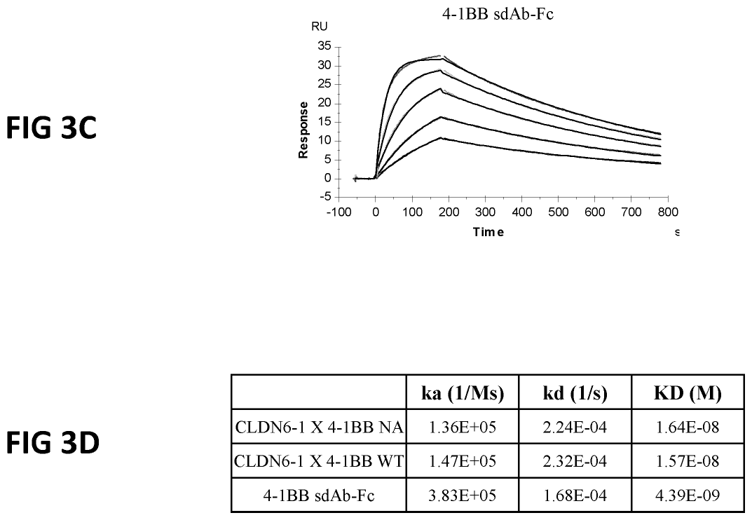

The binding affinity of CLDN6-1×4-1BB NA and CLDN6-1×4-1BB WT towards human 4-1BB was measured by SPR. As shown in FIG. 3A-3D, CLDN6-1×4-1BB NA and CLDN6-1×4-1BB WT bound to monomeric human 4-1BB with K_{D}S of 1.64×10⁻⁸ M and 1.57×10⁻⁸ M, respectively. The affinity of the parental anti-4-1BB sdAb antibody conjugated with IgG1 Fc fragment (4-1BB sdAb-Fc) for 4-1BB was measured in parallel and was found to have a K_{D} of 4.393×10⁻⁹ M, suggesting that the affinity of the 4-1BB antibody moiety in the BsAbs for 4-1BB is comparable to that of the affinity of 4-1BB sdAb-Fc for 4-1BB.

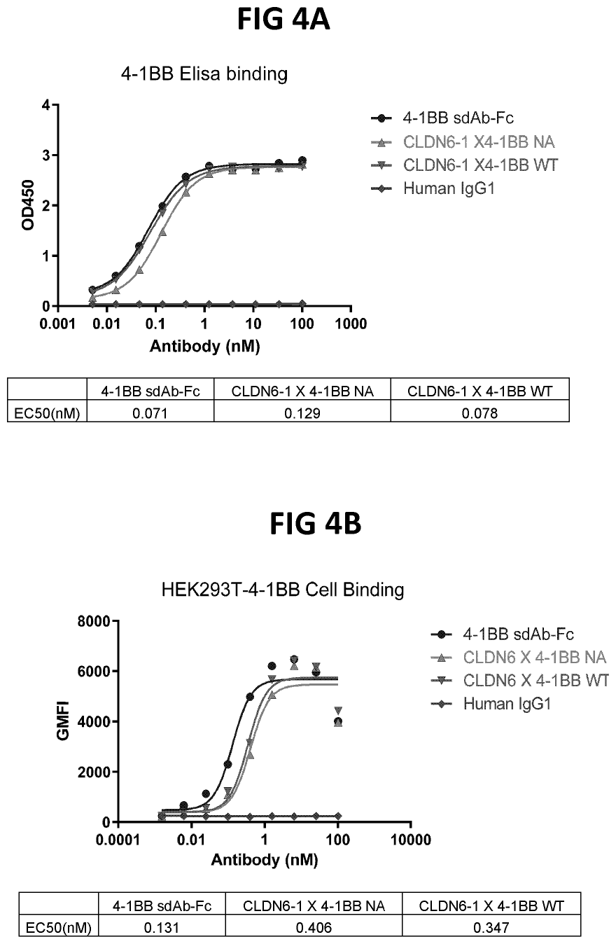

Binding of CLDN6-1×4-1BB NA and CLDN6-1×4-1BB WT with soluble recombinant human 4-1BB was analyzed via ELISA. As shown in FIG. 4A, CLDN6-1×4-1BB NA and CLDN6-1×4-1BB WT both bound to recombinant human 4-1BB in a concentration-dependent manner, with EC50 s of 0.129 nM and 0.078 nM, respectively. Such EC50 s were comparable with the EC50 of 4-1BB sdAb-Fc. In addition, the binding of CLDN6-1×4-1BB NA and CLDN6-1×4-1BB WT with HEK293 cells expressing 4-1BB was evaluated by FACS. As shown in FIG. 4B, CLDN6-1×4-1BB NA and CLDN6-1×4-1BB WT were both capable of binding to 4-1BB with EC50 s of 0.406 nM and 0.347 nM, respectively. Such EC50 s were comparable with the EC50 of 4-1BB sdAb-Fc.

### Example 3. Functional Activity of CLDN6-1×4-1BB BsAbs

### 3.1 Cell-Line Based Functional Characterization of CLDN6×4-1BB BsAbs

To test the ability of the CLDN6×4-1BB bispecific antibodies to activate 4-1BB signal, a GloResponse™ NFκB-luc2/4-1BB Jurkat cell line stably expressing 4-1BB and NFκB luciferase reporter was used as effector cells and CLDN6-expressing cells (CHO-K1 CLDN6, OVCAR3 or OV90) were used as target cells. RKO colon carcinoma cells, which do not express CLDN6, were used as negative control.

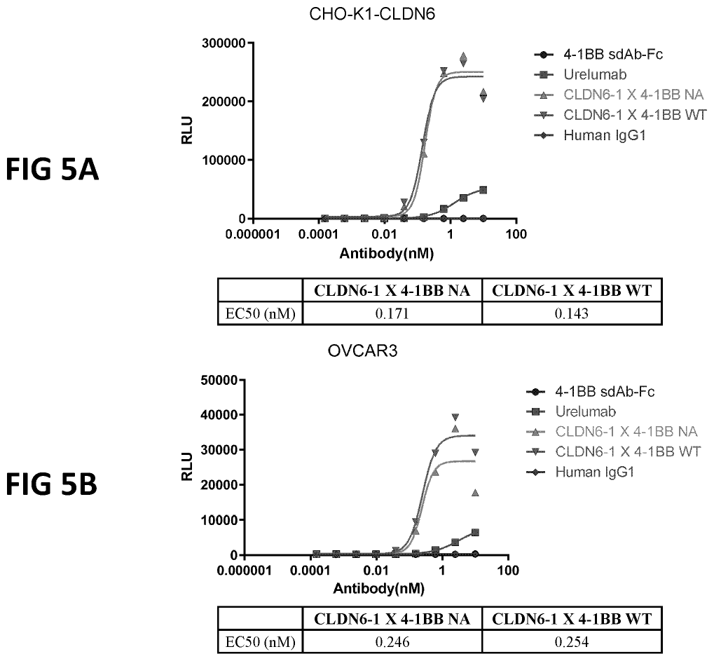

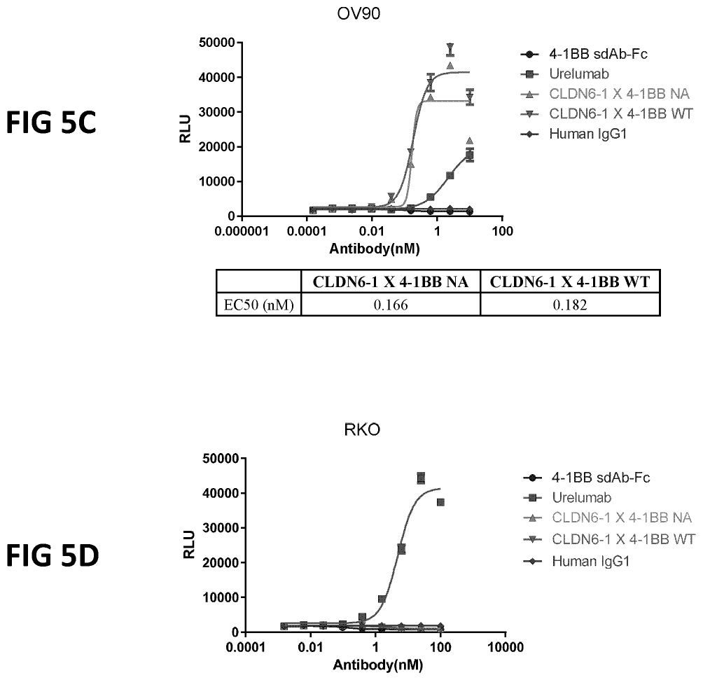

In brief, GloResponse™ NFκB-luc2/4-1BB Jurkat cells (at a density of 5.0×10⁴ cells per well) were mixed with 5.0×10⁴ target cells in a white 96-well plate. Antibodies were serially diluted and added to the plate. Luminescence was measured after 6-hour incubation at 37° C. As shown in FIG. 5A to 5D, urelumab triggered 4-1BB activation regardless of CLDN6 expression, while 4-1BB sdAb-Fc of the present application had no agonist activity under the same experimental setting, despite its being able to bind to 4-1BB. Similarly, CLDN6-1×4-1BB NA and CLDN6-1×4-1BB WT induced NFκB activity in the presence of all CLDN6-expressing target cells, irrespective of CLDN6 expression levels. In contrast, when RKO cells, which do not express CLDN6, were used as target cells, CLDN6-1×4-1BB NA and CLDN6-1×4-1BB WT resulted in significantly lower 4-1BB activation as compared to urelumab, as shown in FIG. 5D.

### 3.2 Activity of CLDN6×4-1BB BsAbs in Promoting Human Peripheral Blood Mononuclear Cell (PBMC) Immune Response

Pre-activated human PBMCs were cocultured with CLDN6 expressing cell or RKO at an effector-to-target (E:T) ratio of 10:1. Antibodies at different concentrations were added to the mixed culture. After 48 hours, the level of IL-2 or IFNγ in culture medium was measured using homogeneous HTRF assay.

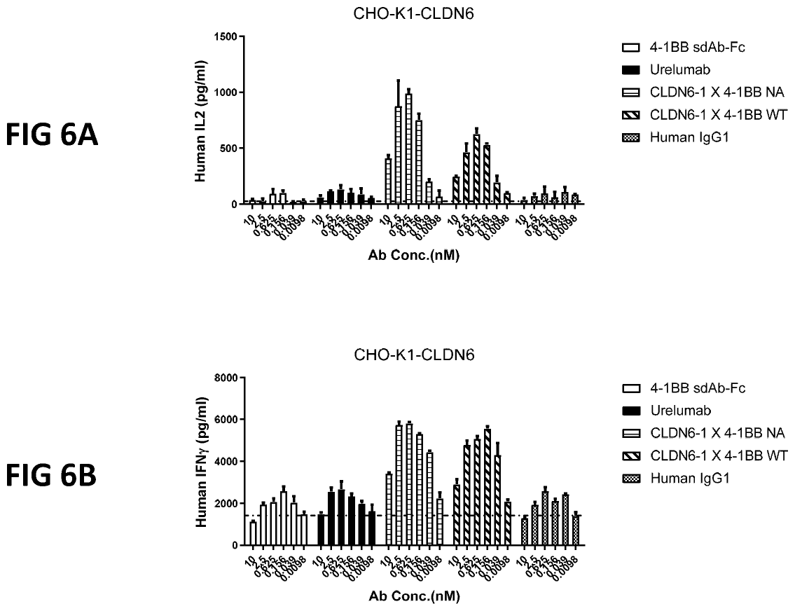

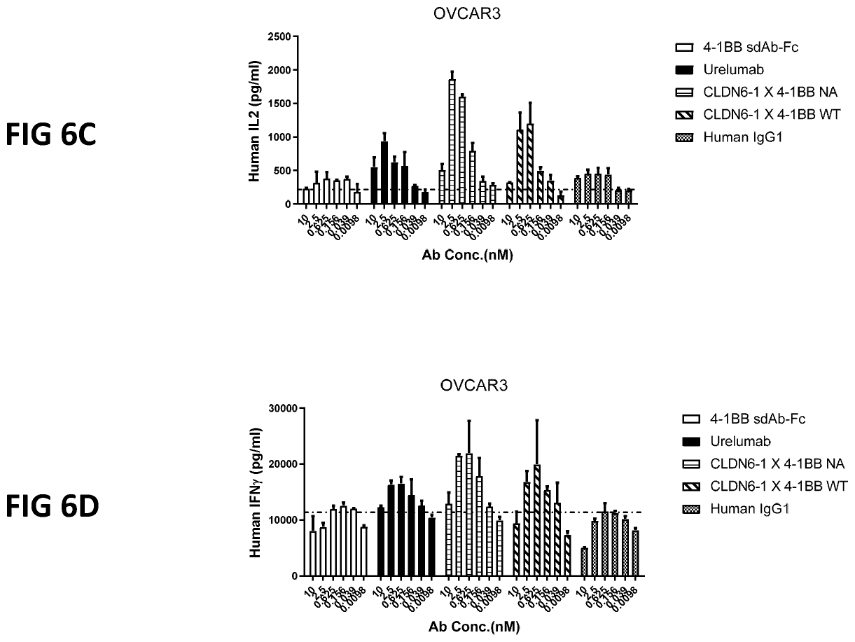

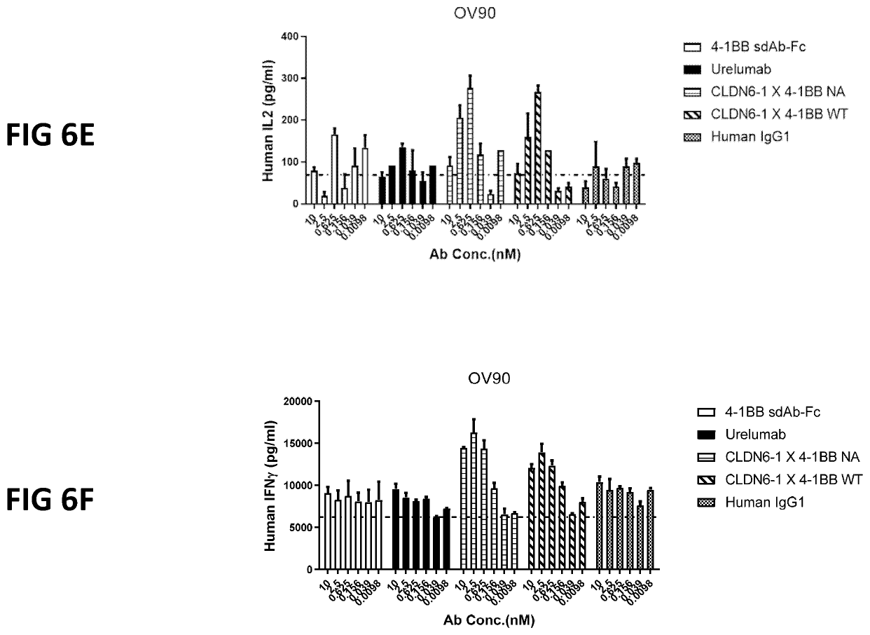

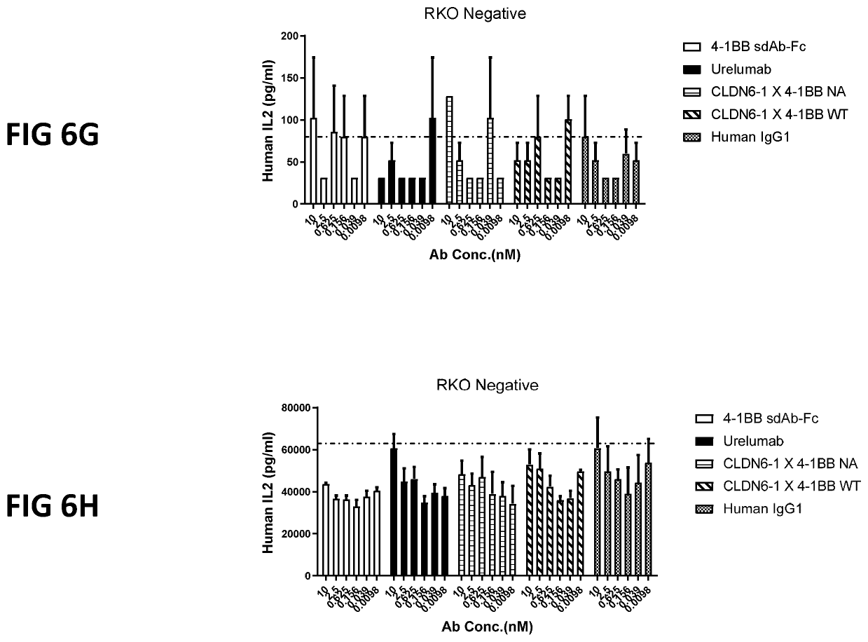

As shown in FIG. 6A-6F, CLDN6-1×4-1BB NA and CLDN6-1×4-1BB WT stimulated IL-2 and IFNγ production when PBMCs were cocultured with CLDN6-expressing target cells. However, in the presence of RKO that do not express CLDN6, CLDN6-1×4-1BB NA and CLDN6-1×4-1BB WT did not stimulate IL-2 or IFNγ production from PBMCs, as shown in FIGS. 6G and 6H, suggesting that the activity of CLDN6-1×4-1BB NA and CLDN6-1×4-1BB WT was dependent on the presence of tumor antigen. In contrast, 4-1BB sdAb-Fc was inactive in this assay.

### Example 4. Tumor Growth Inhibition by CLDN6-1×4-1BB BsAbs

### 4.1 CT26 Syngeneic Murine Colorectal Carcinoma Model

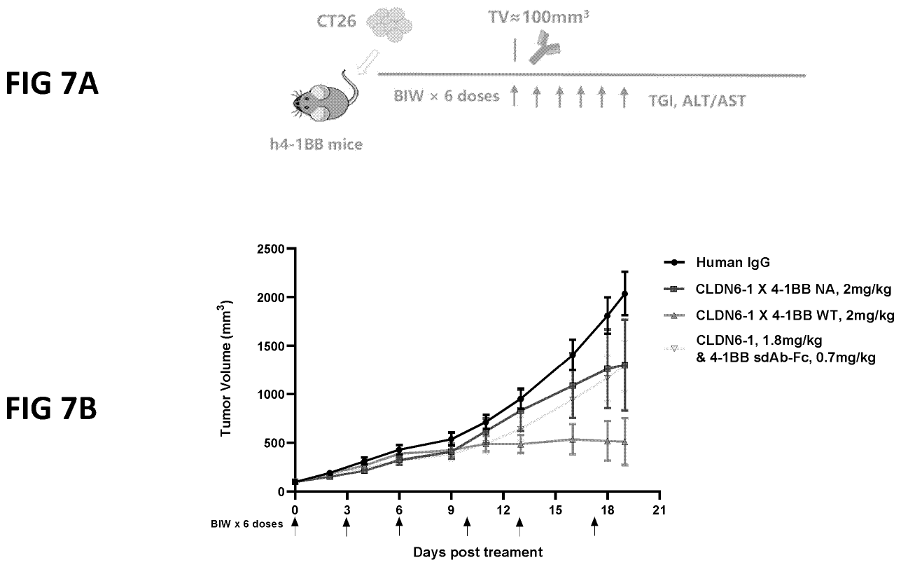

CT26 is an N-nitroso-N-methylurethane-(NNMU) induced undifferentiated colon carcinoma cell line established from BALB/c mice with aggressive colon carcinoma. CT26 cells, which endogenously express CLDN6, were subcutaneously implanted into BALB/c humanized 4-1BB mice. When tumors grew to an average of 100 mm³, the mice were intraperitoneally treated with (a) human IgG, (b) CLDN6-1×4-1BB NA (2 mg/kg), (c) CLDN6-1×4-1BB WT (2 mg/kg), or (d) a combination of the parental CLDN6-1 antibody and the 4-1BB sdAb-Fc (1.8 mg/kg and 0.7 mg/kg). The treatments were administered twice weekly, for a total of 6 doses. Tumor growth was monitored by volumetric measurement. As shown in FIGS. 7A and 7B, CLDN6-1×4-1BB WT and CLDN6-1×4-1BB NA both exhibited anti-tumor activity, in which CLDN6-1×4-1BB WT achieved even stronger activity with tumor growth inhibition (TGI) of 75%.

### 4.2 MC38 Syngeneic Murine Colorectal Adenocarcinoma Model

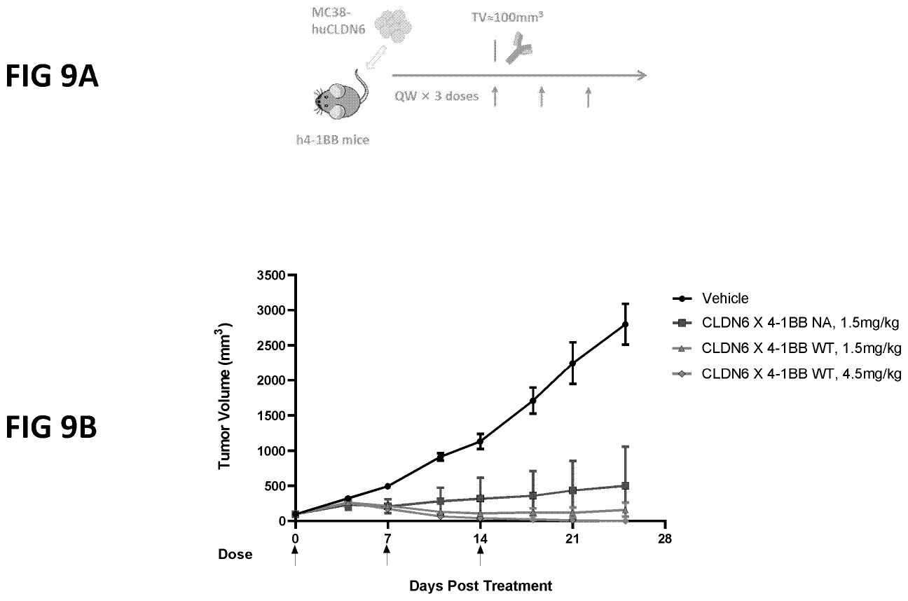

The MC38 tumorigenic epithelial cell line was isolated from mice with colon adenocarcinoma. MC38 cells, which were engineered to express human CLDN6, were subcutaneously implanted into C57BL/6 humanized 4-1BB mice. When tumors grew to an average of 100 mm³, the mice were intraperitoneally treated with (a) vehicle (control), (b) CLDN6-1×4-1BB NA (1.5 mg/kg), (c) CLDN6-1×4-1BB WT (1.5 mg/kg), or (d) CLDN6-1×4-1BB WT (4.5 mg/kg). The treatments were administered weekly, for a total of 3 doses. See FIG. 9A. Tumor growth was monitored by volumetric measurement. As shown in FIG. 9B, CLDN6-1×4-1BB NA exhibited significant anti-tumor activity, whereas treatment with CLDN6-1×4-1BB WT achieved more significant tumor growth inhibition under the same dose, and led to complete tumor regression under higher dose.

### Example 5. Liver Toxicity Evaluation of CLDN6×4-1BB BsAbs

The major concern of t4-1BB agonist antibody therapy is dose-limiting liver toxicity, as observed in urelumab's clinical development. Most common adverse events were elevated alanine transaminase (ALT), aspartate aminotransferase (AST) and fatigue. Thus, the liver toxicity of CLDN6-1×4-1BB WT and CLDN6-1×4-1BB NA was further evaluated.

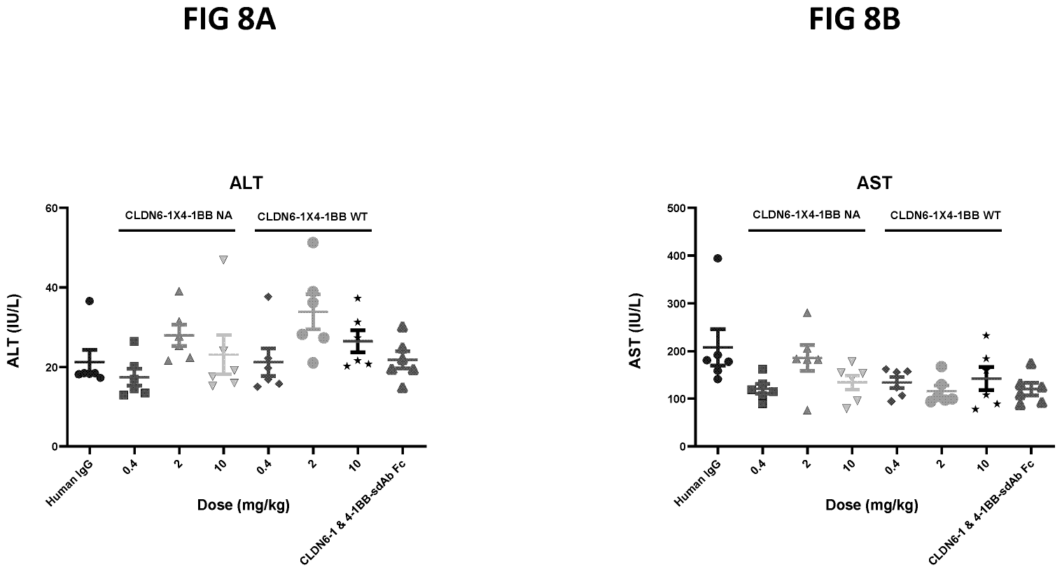

Briefly, blood samples were collected for ALT and AST measurements from hu4-1BB mice after treatment of CLDN6-1×4-1BB WT or CLDN6-1×4-1BB NA at different doses twice weekly. As shown in FIG. 8, no significant elevation of ALT and AST was observed, suggesting little risk for liver toxicity commonly induced by other 4-1BB agonist antibodies.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Summary Table of Amino Acid Sequences

| SEQ ID | | |
|---|---|---|
| NO: | SEQUENCE | DESCRIPTION |
| 1 | SGYYWN | CLDN6-1 |
| | | VH-CDR1 |
| 2 | YISYSGSNNYNPSLKN | CLDN6-1 |
| | | VH-CDR2 |
| 3 | GGYYFDY | CLDN6-1 |
| | | VH-CDR3 |
| 4 | KSSQSLLNTNNQKNYLA | CLDN6-1 |
| | | VL-CDR1 |
| 5 | FASTRES | CLDN6-1 |
| | | VL-CDR2 |
| 6 | QQHYNAPRT | CLDN6-1 |
| | | VL-CDR3 |
| 7 | QVQLQESGPGLVKPSETLSLTCTVSGYSITSGYYWNWIRQPPGKGLEW | CLDN6-1 |
| | MGYISYSGSNNYNPSLKNRVTISRDTSKNQFSLKLSSVTAADTAVYYC | VH |
| | ARGGYYFDYWGQGTLVTVSS | |
| 8 | DIVMTQSPDSLAVSLGERATINCKSSQSLLNTNNQKNYLAWYQQKPG | CLDN6-1 |
| | QPPKLLIYFASTRESGVPDRFSGSGSGTDFTLTISSLQAEDVAVYFCQQH | VL |
| | YNAPRTFGGGTKVEIK | |
| 9 | QVQLQESGPGLVKPSETLSLTCTVSGYSITSGYYWNWIRQPPGKGLEW | HC of CLDN6-1 |
| | MGYISYSGSNNYNPSLKNRVTISRDTSKNQFSLKLSSVTAADTAVYYC | WT |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGK | |
| 10 | QVQLQESGPGLVKPSETLSLTCTVSGYSITSGYYWNWIRQPPGKGLEW | HC of CLDN6-1 |
| | MGYISYSGSNNYNPSLKNRVTISRDTSKNQFSLKLSSVTAADTAVYYC | N297A |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYASTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGK | |
| 11 | DIVMTQSPDSLAVSLGERATINCKSSQSLLNTNNQKNYLAWYQQKPG | LC of CLDN6-1 |
| | QPPKLLIYFASTRESGVPDRFSGSGSGTDFTLTISSLQAEDVAVYFCQQH | |
| | YNAPRTFGGGTKVEIKRTVAAPSVFIFPPSDEQLKSGTASVVCLLNNFY | |
| | PREAKVQWKVDNALQSGNSQESVTEQDSKDSTYSLSSTLTLSKADYE | |
| | KHKVYACEVTHQGLSSPVTKSFNRGEC | |
| 12 | IYGMS | CLDN6-3 |
| | | VH-CDR1 |
| 13 | TISRDASYTYFPDSVKG | CLDN6-3 |
| | | VH-CDR2 |
| 14 | LGDNDRGYAMDY | CLDN6-3 |
| | | VH-CDR3 |
| 15 | RSSQSVVHVNANTYLE | CLDN6-3 |
| | | VL-CDR1 |
| 16 | KVSNRFP | CLDN6-3 |
| | | VL-CDR2 |
| 17 | FQGSHVPRT | CLDN6-3 |
| | | VL-CDR3 |
| 18 | EVQLVESGGGLVQPGGSLRLSCAASGFTFSIYGMSWVRQAPGKGLEW | CLDN6-3 |
| | VATISRDASYTYFPDSVKGRFTISRDNAKNSLYLQMNSLRAEDTAVYY | VH |
| | CVRLGDNDRGYAMDYWGQGTLVTVSS | |
| 19 | DVVMTQSPLSLPVTLGQPASISCRSSQSVVHVNANTYLEWYQQRPGQS | CLDN6-3 |
| | PRLLIYKVSNRFPGVPDRFSGSGSGTDFTLKISRVEAEDVGVYYCFQGS | VL |
| | HVPRTFGGGTKVEIK | |
| 20 | EVQLVESGGGLVQPGGSLRLSCAASGFTFSIYGMSWVRQAPGKGLEW | HC of CLDN6-3 |
| | VATISRDASYTYFPDSVKGRFTISRDNAKNSLYLQMNSLRAEDTAVYY | WT |
| | CVRLGDNDRGYAMDYWGQGTLVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSC | |
| | SVMHEALHNHYTQKSLSLSPGK | |
| 21 | EVQLVESGGGLVQPGGSLRLSCAASGFTFSIYGMSWVRQAPGKGLEW | HC of CLDN6-3 |
| | VATISRDASYTYFPDSVKGRFTISRDNAKNSLYLQMNSLRAEDTAVYY | N297A |
| | CVRLGDNDRGYAMDYWGQGTLVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYASTYRVVSVLTVLHQDWLNGKEYKCKVSNKALP | |
| | APIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSC | |
| | SVMHEALHNHYTQKSLSLSPGK | |
| 22 | DVVMTQSPLSLPVTLGQPASISCRSSQSVVHVNANTYLEWYQQRPGQS | LC of CLDN6-3 |
| | PRLLIYKVSNRFPGVPDRFSGSGSGTDFTLKISRVEAEDVGVYYCFQGS | |
| | HVPRTFGGGTKVEIKRTVAAPSVFIFPPSDEQLKSGTASVVCLLNNFYP | |
| | REAKVQWKVDNALQSGNSQESVTEQDSKDSTYSLSSTLTLSKADYEK | |
| | HKVYACEVTHQGLSSPVTKSFNRGEC | |
| 23 | GGGGSGGGGSGGGGS | Linker |
| 24 | SNCMG | 4-1BB CDR1 |
| 25 | VICTGGGSPSYADSVKG | 4-1BB CDR2 |
| 26 | DLLRAGTPLSSYEFNY | 4-1BB CDR3 |
| 27 | QVQLQESGGGLVQPGGSLRLSCAASGYTYSSNCMGWFRQAPGKGLEG | 4-1BB |
| | VAVICTGGGSPSYADSVKGRFTISRDNAKNTLYLQMNSLRAEDTAVY | VHH |
| | YCAADLLRAGTPLSSYEFNYWGQGTLVTVSS | |
| 28 | QVQLQESGPGLVKPSETLSLTCTVSGYSITSGYYWNWIRQPPGKGLEW | Heavy chain |
| | MGYISYSGSNNYNPSLKNRVTISRDTSKNQFSLKLSSVTAADTAVYYC | component |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYASTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGKGGGGSGGGGSGGGGSQVQLQESGGGLVQPGGSL | |
| | RLSCAASGYTYSSNCMGWFRQAPGKGLEGVAVICTGGGSPSYADSVK | |
| | GRFTISRDNAKNTLYLQMNSLRAEDTAVYYCAADLLRAGTPLSSYEFN | |
| | YWGQGTLVTVSS | |
| 29 | DIVMTQSPDSLAVSLGERATINCKSSQSLLNTNNQKNYLAWYQQKPG | Light chain |
| | QPPKLLIYFASTRESGVPDRFSGSGSGTDFTLTISSLQAEDVAVYFCQQH | component |
| | YNAPRTFGGGTKVEIKRTVAAPSVFIFPPSDEQLKSGTASVVCLLNNFY | |
| | PREAKVQWKVDNALQSGNSQESVTEQDSKDSTYSLSSTLTLSKADYE | |
| | KHKVYACEVTHQGLSSPVTKSFNRGEC | |
| 30 | QVQLQESGPGLVKPSETLSLTCTVSGYSITSGYYWNWIRQPPGKGLEW | Heavy chain |
| | MGYISYSGSNNYNPSLKNRVTISRDTSKNQFSLKLSSVTAADTAVYYC | component |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGKGGGGSGGGGSGGGGSQVQLQESGGGLVQPGGSL | |
| | RLSCAASGYTYSSNCMGWFRQAPGKGLEGVAVICTGGGSPSYADSVK | |
| | GRFTISRDNAKNTLYLQMNSLRAEDTAVYYCAADLLRAGTPLSSYEFN | |
| | YWGQGTLVTVSS | |
| 31 | DIVMTQSPDSLAVSLGERATINCKSSQSLLNTNNQKNYLAWYQQKPG | Light chain |
| | QPPKLLIYFASTRESGVPDRFSGSGSGTDFTLTISSLQAEDVAVYFCQQH | component |
| | YNAPRTFGGGTKVEIKRTVAAPSVFIFPPSDEQLKSGTASVVCLLNNFY | |
| | PREAKVQWKVDNALQSGNSQESVTEQDSKDSTYSLSSTLTLSKADYE | |
| | KHKVYACEVTHQGLSSPVTKSFNRGEC | |
| 32 | QVQLQESGGGLVQPGGSLRLSCAASGYTYSSNCMGWFRQAPGKGLEG | Heavy chain |
| | VAVICTGGGSPSYADSVKGRFTISRDNAKNTLYLQMNSLRAEDTAVY | component |
| | YCAADLLRAGTPLSSYEFNYWGQGTLVTVSSGGGGSGGGGSGGGGSQ | |
| | VQLQESGPGLVKPSETLSLTCTVSGYSITSGYYWNWIRQPPGKGLEWM | |
| | GYISYSGSNNYNPSLKNRVTISRDTSKNQFSLKLSSVTAADTAVYYCA | |
| | RGGYYFDYWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGT | |
| | QTYICNVNHKPSNTKVDKKVEPKSCDKTHTCPPCPAPELLGGPSVFLFP | |
| | PKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKA | |
| | KGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHN | |
| | HYTQKSLSLSPGK | |
| 33 | DIVMTQSPDSLAVSLGERATINCKSSQSLLNTNNQKNYLAWYQQKPG | Light chain |
| | QPPKLLIYFASTRESGVPDRFSGSGSGTDFTLTISSLQAEDVAVYFCQQH | component |
| | YNAPRTFGGGTKVEIKRTVAAPSVFIFPPSDEQLKSGTASVVCLLNNFY | |
| | PREAKVQWKVDNALQSGNSQESVTEQDSKDSTYSLSSTLTLSKADYE | |
| | KHKVYACEVTHQGLSSPVTKSFNRGEC | |
| 34 | QVQLQESGPGLVKPSETLSLTCTVSGYSITSGYYWNWIRQPPGKGLEW | Heavy chain |
| | MGYISYSGSNNYNPSLKNRVTISRDTSKNQFSLKLSSVTAADTAVYYC | component |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGK | |
| 35 | QVQLQESGGGLVQPGGSLRLSCAASGYTYSSNCMGWFRQAPGKGLEG | Light chain |
| | VAVICTGGGSPSYADSVKGRFTISRDNAKNTLYLQMNSLRAEDTAVY | component |
| | YCAADLLRAGTPLSSYEFNYWGQGTLVTVSSGGGGSGGGGSGGGGSD | |
| | IVMTQSPDSLAVSLGERATINCKSSQSLLNTNNQKNYLAWYQQKPGQP | |
| | PKLLIYFASTRESGVPDRFSGSGSGTDFTLTISSLQAEDVAVYFCQQHY | |
| | NAPRTFGGGTKVEIKRTVAAPSVFIFPPSDEQLKSGTASVVCLLNNFYP | |
| | REAKVQWKVDNALQSGNSQESVTEQDSKDSTYSLSSTLTLSKADYEK | |
| | HKVYACEVTHQGLSSPVTKSFNRGEC | |
| 36 | EVQLVESGGGLVQPGGSLRLSCAASGFTFSIYGMSWVRQAPGKGLEW | Heavy chain |
| | VATISRDASYTYFPDSVKGRFTISRDNAKNSLYLQMNSLRAEDTAVYY | component |
| | CVRLGDNDRGYAMDYWGQGTLVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYASTYRVVSVLTVLHQDWLNGKEYKCKVSNKALP | |
| | APIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSC | |
| | SVMHEALHNHYTQKSLSLSPGKGGGGSGGGGSGGGGSQVQLQESGG | |
| | GLVQPGGSLRLSCAASGYTYSSNCMGWFRQAPGKGLEGVAVICTGGG | |
| | SPSYADSVKGRFTISRDNAKNTLYLQMNSLRAEDTAVYYCAADLLRA | |
| | GTPLSSYEFNYWGQGTLVTVSS | |
| 37 | DVVMTQSPLSLPVTLGQPASISCRSSQSVVHVNANTYLEWYQQRPGQS | Light chain |
| | PRLLIYKVSNRFPGVPDRFSGSGSGTDFTLKISRVEAEDVGVYYCFQGS | component |
| | HVPRTFGGGTKVEIKRTVAAPSVFIFPPSDEQLKSGTASVVCLLNNFYP | |
| | REAKVQWKVDNALQSGNSQESVTEQDSKDSTYSLSSTLTLSKADYEK | |
| | HKVYACEVTHQGLSSPVTKSFNRGEC | |
| 38 | EVQLVESGGGLVQPGGSLRLSCAASGFTFSIYGMSWVRQAPGKGLEW | Heavy chain |
| | VATISRDASYTYFPDSVKGRFTISRDNAKNSLYLQMNSLRAEDTAVYY | component |
| | CVRLGDNDRGYAMDYWGQGTLVTVSSASTKGPSVFPLAPSSKSTSGG | |
| | TAALGCLVKDYFPEPVTVSWNSGALTSGVHTFPAVLQSSGLYSLSSVV | |
| | TVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSC | |
| | SVMHEALHNHYTQKSLSLSPGKGGGGSGGGGSGGGGSQVQLQESGG | |
| | GLVQPGGSLRLSCAASGYTYSSNCMGWFRQAPGKGLEGVAVICTGGG | |
| | SPSYADSVKGRFTISRDNAKNTLYLQMNSLRAEDTAVYYCAADLLRA | |
| | GTPLSSYEFNYWGQGTLVTVSS | |
| 39 | DVVMTQSPLSLPVTLGQPASISCRSSQSVVHVNANTYLEWYQQRPGQS | Light chain |
| | PRLLIYKVSNRFPGVPDRFSGSGSGTDFTLKISRVEAEDVGVYYCFQGS | component |
| | HVPRTFGGGTKVEIKRTVAAPSVFIFPPSDEQLKSGTASVVCLLNNFYP | |
| | REAKVQWKVDNALQSGNSQESVTEQDSKDSTYSLSSTLTLSKADYEK | |
| | HKVYACEVTHQGLSSPVTKSFNRGEC | |
| 40 | MASAGMQILGVVLTLLGWVNGLVSCALPMWKVTAFIGNSIVVAQVV | CLDN6 |
| | WEGLWMSCVVQSTGQMQCKVYDSLLALPQDLQAARALCVIALLVAL | |
| | FGLLVYLAGAKCTTCVEEKDSKARLVLTSGIVFVISGVLTLIPVCWTAH | |
| | AIIRDFYNPLVAEAQKRELGASLYLGWAASGLLLLGGGLLCCTCPSGG | |
| | SQGPSHYMARYSTSAPAISRGPSEYPTKNYV | |
| 41 | LQDPCSNCPAGTFCDNNRNQICSPCPPNSFSSAGGQRTCDICRQCKGVF | 4-1BB |
| | RTRKECSSTSNAECDCTPGFHCLGAGCSMCEQDCKQGQELTKKGCKD | |
| | CCFGTFNDQKRGICRPWTNCSLDGKSVLVNGTKERDVVCGPSPADLSP | |
| | GASSVTPPAPAREPGHSPQIISFFLALTSTALLFLLFFLTLRFSVVKRGRK | |
| | KLLYIFKQPFMRPVQTTQEEDGCSCRFPEEEEGGCEL | |
| 42 | QVQLQESGPGLVKPSETLSLTCTVSGYSITSGYYWNWIRQPPGKGLEW | HC of CLDN6-1 |
| | MGYISYSGSNNYNPSLKNRVTISRDTSKNQFSLKLSSVTAADTAVYYC | WT |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSSLSPG | |
| 43 | QVQLQESGPGLVKPSETLSLTCTVSGYSITSGYYWNWIRQPPGKGLEW | HC of CLDN6-1 |
| | MGYISYSGSNNYNPSLKNRVTISRDTSKNQFSLKLSSVTAADTAVYYC | N297A |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYASTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPG | |
| 44 | QVQLQESGPGLVKPSETLSLTCTVSGYSITSGYYWNWIRQPPGKGLEW | Heavy chain |
| | MGYISYSGSNNYNPSLKNRVTISRDTSKNQFSLKLSSVTAADTAVYYC | component |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYASTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGGGGGSGGGGSGGGGSQVQLQESGGGLVQPGGSLR | |
| | LSCAASGYTYSSNCMGWFRQAPGKGLEGVAVICTGGGSPSYADSVKG | |
| | RFTISRDNAKNTLYLQMNSLRAEDTAVYYCAADLLRAGTPLSSYEFNY | |
| | WGQGTLVTVSS | |
| 45 | QVQLQESGPGLVKPSETLSLTCTVSGYSITSGYYWNWIRQPPGKGLEW | Heavy chain |
| | MGYISYSGSNNYNPSLKNRVTISRDTSKNQFSLKLSSVTAADTAVYYC | component |
| | ARGGYYFDYWGQGTLVTVSSASTKGPSVFPLAPSSKSTSGGTAALGCL | |
| | VKDYFPEPVTVSWNSGALTSGVHTFPAVLQSSGLYSLSSVVTVPSSSLG | |
| | TQTYICNVNHKPSNTKVDKKVEPKSCDKTHTCPPCPAPELLGGPSVFLF | |
| | PPKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQ | |
| | PENNYKTTPPVLDSDGSFFLYSKLTVDKSRWQQGNVFSCSVMHEALH | |
| | NHYTQKSLSLSPGGGGGSGGGGSGGGGSQVQLQESGGGLVQPGGSLR | |
| | LSCAASGYTYSSNCMGWFRQAPGKGLEGVAVICTGGGSPSYADSVKG | |
| | RFTISRDNAKNTLYLQMNSLRAEDTAVYYCAADLLRAGTPLSSYEFNY | |
| | WGQGTLVTVSS | |

1. A multispecific construct comprising:
(1) a first antibody moiety that specifically binds to claudin-6 (“CLDN6”); and
(2) a second antibody moiety that specifically binds to 4-1BB.

2. The multispecific construct of claim 0, wherein the first antibody moiety is selected from the group consisting of a full-length antibody, Fab, Fab′, F(ab′)₂, scFv, and sdAb.

3. The multispecific construct of claim 0 or 0, wherein the first antibody moiety comprises:
(1) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 7; and
(2) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID No: 8.

4. The multispecific construct of any one of claims 0-0, wherein the first antibody moiety comprises a heavy variable region (VH) and a light chain variable region (VL), wherein:
(a) the VH comprises:
(i) a HC-CDR1 comprising an amino acid sequence of SEQ ID NO: 1,
(ii) a HC-CDR2 comprising an amino acid sequence of SEQ ID NO: 2, and
(iii) a HC-CDR3 comprising an amino acid sequence of SEQ ID NO: 3, and;
(b) the VL comprises:
(i) a LC-CDR1 comprising an amino acid sequence of SEQ ID NO: 4,
(ii) a LC-CDR2 comprising an amino acid sequence of SEQ ID NO: 5, and
(iii) a LC-CDR3 comprising an amino acid sequence of SEQ ID NO: 6.

5. The multispecific construct of any one of claims 0-0, wherein the first antibody moiety comprises a heavy variable region (VH) and a light chain variable region (VL), and wherein:
(1) the VH comprises the amino acid sequence of SEQ ID NO: 7, or a variant thereof comprising at least about 80% sequence identity to SEQ ID NO: 7; and/or
(2) the VL comprises the amino acid sequence of SEQ ID NO: 8, or a variant thereof comprising at least about 80% sequence identity to SEQ ID NO: 8.

6. The multispecific construct of any one of claims 0-2, wherein the first antibody moiety comprises:
(1) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 18; and
(2) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID NO: 19.

7. The multispecific construct of any one of claims 0-2 and 6, wherein the first antibody moiety comprises a heavy variable region (VH) and a light chain variable region (VL), wherein:
(1) the VH comprises:
(i) a HC-CDR1 comprising an amino acid sequence of SEQ ID NO: 12,
(ii) a HC-CDR2 comprising an amino acid sequence of SEQ ID NO: 13, and
(iii) a HC-CDR3 comprising an amino acid sequence of SEQ ID NO: 14, and;
(2) the VL comprises:
(i) a LC-CDR1 comprising an amino acid sequence of SEQ ID NO: 15,
(ii) a LC-CDR2 comprising an amino acid sequence of SEQ ID NO: 16, and
(iii) a LC-CDR3 comprising an amino acid sequence of SEQ ID NO: 17.

8. The multispecific construct of any one of claims 1-2 and 6-7, wherein the first antibody moiety comprises a heavy variable region (VH) and a light chain variable region (VL), and wherein:
(1) the VH comprises the amino acid sequence of SEQ ID NO: 18, or a variant thereof having at least about 80% sequence identity to SEQ ID NO: 18; and/or
(2) the VL comprises the amino acid sequence of SEQ ID NO: 19, or a variant thereof having at least about 80% sequence identity to SEQ ID NO: 19.

9. The multispecific construct of any one of claims 0-8, wherein binding of the first antibody moiety with CLDN6 triggers the second antibody moiety to activate 4-1BB.

10. The multispecific construct of claim 9, wherein the second antibody moiety specifically binds to CRD 3/4 region of 4-1BB.

11. The multispecific construct of any one of claims 0-10, wherein the second antibody moiety is selected from the group consisting of a full-length antibody, Fab, Fab′, F(ab′)₂, scFv, and sdAb.

12. The multispecific construct of claim 11, wherein the second antibody moiety is a sdAb.

13. The multispecific construct of claim 12, wherein the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27.

14. The multispecific construct of claim 12, wherein the sdAb comprises:
(1) a sdAb-CDR1 comprising an amino acid sequence of SEQ ID NO: 24;
(2) a sdAb-CDR2 comprising an amino acid sequence of SEQ ID NO: 25; and
(3) a sdAb-CDR3 comprising an amino acid sequence of SEQ ID NO: 26.

15. The multispecific construct of any one of claims 0-14, wherein the second antibody moiety comprises the amino acid sequence of SEQ ID NO:27, or a variant thereof having at least about 80% sequence identity to SEQ ID NOs: 27.

16. The multispecific construct of any one of claims 0-15, wherein the multispecific construct is a bispecific antibody or a bispecific binding fragment.

17. The multispecific construct of any one of claims 0-5 and 9-16, wherein:
(1) the first antibody moiety comprises:
(a) a HC-CDR1, a HC-CDR2, and a HC-CDR3, respectively comprising the amino acid sequences of a CDR1, a CDR2, and a CDR3 within a heavy variable region (VH) comprising the sequence set forth in SEQ ID No: 7;
(b) a LC-CDR1, a LC-CDR2, and a LC-CDR3, respectively comprising the amino acid sequences of a CDR1, a CDR2, and a CDR3 within a light chain variable region (VL) comprising the sequence set forth in SEQ ID No: 8, and
(2) the second antibody moiety comprises a sdAb-CDR1, a sdAb-CDR2, and a sdAb CDR3, respectively comprising the amino acid sequence of a CDR1, a CDR2, and a CDR3 within a single monomeric variable antibody domain comprising the amino acid sequence set forth in SEQ ID NOs: 27.

18. A pharmaceutical composition comprising the multispecific construct of any one of claims 0-17, and a pharmaceutical acceptable carrier.

19. A nucleic acid encoding the multispecific construct of any one of claims 0-17.

20. A vector comprising the nucleic acid of claim 19.

21. A host cell comprising the nucleic acid of claim 19, or the vector of claim 20.

22. A method of treating a disease or condition in a subject in need thereof, comprising administering to the subject an effective amount of the multispecific construct of any one of claims 0-17, or the pharmaceutical composition of claim 18.

23. The method of claim 22, wherein the disease or condition is cancer.

24. Use of the multispecific construct of any one of claims 0-17 in preparing a medicament for treating a disease or condition in a subject in need thereof.

25. A multispecific construct comprising:
(1) a first antibody moiety that specifically binds to a tumor antigen; and
(2) a second antibody moiety that specifically binds to 4-1BB, wherein binding of the first antibody moiety with the tumor antigen triggers the second antibody moiety to activate 4-1BB.

26. The multispecific construct of claim 0, wherein the activation of 4-1BB by the second antibody moiety is enhanced by at least 10 folds after binding of the first antibody moiety with the tumor antigen.

27. The multispecific construct of claim 0 or 0, wherein the second antibody moiety specifically binds to CRD 3/4 region of 4-1BB.

28. The multispecific construct of any one of claims 0-0, wherein the second antibody moiety is a sdAb.

29. The multispecific construct of claim 0, wherein the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27.

30. The multispecific construct of claim 0, wherein the sdAb comprises:
(1) a sdAb-CDR1 comprising an amino acid sequence of SEQ ID NO: 24;
(2) a sdAb-CDR2 comprising an amino acid sequence of SEQ ID NO: 25; and
(3) a sdAb-CDR3 comprising an amino acid sequence of SEQ ID NO: 26.

31. The multispecific construct of any one of claims 0-0, wherein the tumor antigen is CLDN6.

32. A multispecific construct, comprising two heavy chain components and two light chain components, wherein:
(a) each heavy chain component comprises a sequence set forth in SEQ ID NO: 28 and each light chain component comprises a sequence set forth in SEQ ID NO: 29;
(b) each heavy chain component comprises a sequence set forth in SEQ ID NO: 30 and each light chain component comprises a sequence set forth in SEQ ID NO: 31;
(c) each heavy chain component comprises a sequence set forth in SEQ ID NO: 32 and each light chain component comprises a sequence set forth in SEQ ID NO: 33;
(d) each heavy chain component comprises a sequence set forth in SEQ ID NO: 34 and each light chain component comprises a sequence set forth in SEQ ID NO: 35;
(e) each heavy chain component comprises a sequence set forth in SEQ ID NO: 36 and each light chain component comprises a sequence set forth in SEQ ID NO: 37; or
(f) each heavy chain component comprises a sequence set forth in SEQ ID NO: 38 and each light chain component comprises a sequence set forth in SEQ ID NO: 39.
